SEQUENCE LISTING (2 sequences)

SEQ ID NO: 1 - protein, 36 aa, synthetic construct
MCMPCFTTDHQMARKCDDCCGGKGRGKCYGPQCLCR

SEQ ID NO: 2 - protein, 36 aa, synthetic construct; identical to SEQ ID NO: 1
  DISULFID 2..19: note = Intrachain, between Cys2 and Cys19
  DISULFID 5..28: note = Intrachain, between Cys5 and Cys28
  DISULFID 16..33: note = Intrachain, between Cys16 and Cys33
  DISULFID 20..35: note = Intrachain, between Cys20 and Cys35

(19) United States
(12) Patent Application Publication
(10) Pub. No.: US 2026/0014272 A1
(43) Pub. Date: Jan. 15, 2026

(54) GENERATION METHODS OF CHIMERIC ANTIGEN RECEPTOR ENGINEERED EXTRACELLULAR VESICLES

(71) Applicant: The Florida State University Research Foundation, Inc., Tallahassee, FL (US)

(72) Inventor: Yan Li, Tallahassee, FL (US)

(21) Appl. No.: 19/265,025

(22) Filed: Jul. 10, 2025

Related U.S. Application Data
(60) Provisional application No. 63/669,441, filed on Jul. 10, 2024.

(51) Int. Cl. A61K 47/69; A61K 47/54; A61K 47/64; A61P 35/00

(52) U.S. Cl. CPC A61K 47/6901; A61K 47/544; A61K 47/6425; A61P 35/00

(57) ABSTRACT

Disclosed herein are engineered neutrophil extracellular vesicles comprising a cancer- or tumor cell-targeting Chimeric antigen receptor, a therapeutic composition, and at least one miRNA. Also disclosed herein are methods of reducing or inhibiting growth of a cancer or tumor by administering to a subject positive for the cancer or tumor the engineered neutrophil extracellular vesicles disclosed herein.

### CROSS REFERENCE TO RELATED APPLICATIONS

This application claims the benefit of U.S. Provisional Patent Application 63/669,441, filed Jul. 10, 2024, the disclosure of which is hereby incorporated by reference in its entirety.

### FIELD OF THE INVENTION

The present invention relates to engineered neutrophil extracellular vesicle compositions and uses thereof in a method of reducing or inhibiting growth of a cancer or a tumor.

### INCORPORATION BY REFERENCE

The contents of the xml file named “10850-106US1-ST26” which was created on Jun. 23, 2025, and is 4.23 KB in size, are hereby incorporated by reference in their entirety.

### BACKGROUND OF THE INVENTION

Glioblastoma (GBM) is the most devasting malignant brain cancer in adults and children. Patients bearing GBMs have poor prognosis and high disability rates despite standard of care and poor quality of life due to the side effects of chemoradiation. Lack of effective therapies can be partly explained by the presence of a blood-brain barrier (BBB), the infiltrative nature of GBMs, and a complex immune tumor biology with multiple interconnected pathways and feedback loops. Despite numerous efforts to target critical pathways such as programmed cell death ligand 1 (PD-L1), these approaches have not been successful to date in improving patient outcomes due to factors such as inefficient delivery, targeting specificity, and tumor resistance.

PD-L1 and Lamin B (LMNB) have attracted great interest in the GBM field given the reports on the contribution of their overexpression to aggressiveness of GBMs, leading to suggestions on downregulation of PD-L1 and LMNB reducing tumor growth. Numerous trials have been completed on single drug strategies targeting PD1/PD-L1 axis in GBM with limited success, in part due to T-Cell exhaustion.

Neutrophils (NPs) are the most abundant innate immune cells in human circulation. NPs have a short half-life and are resistant to genetic modifications. Just recently, NPs were engineered by chimeric antigen receptor (CAR) to enhance their anti-tumor cytotoxicity for targeted immunotherapy, representing an exciting new avenue for targeted immunotherapy. CAR exosomes do not express PD1 like their parent cells and have shown more potent anti-tumor effects and low toxicity compared to the parent CAR cells. The CAR-containing exosomes express a high level of cytotoxic molecules and better inhibit tumor growth, being safer than the CAR cells. However, the properties of EVs secreted by CAR-iPSC-neutrophils have not been well studied for cancer/tumor treatment.

Thus, there is an urgent need to address the aforementioned problems and other shortcomings associated with traditional treatment methods of cancer/tumor, such as, GBM.

### SUMMARY OF THE INVENTION

The present invention relates to an engineered neutrophil extracellular vesicle (NPEV) and use thereof in a method of reducing or inhibiting growth of a cancer or a tumor.

In one aspect, the present invention provides an engineered NPEV comprising a cancer- or tumor-targeting Chimeric antigen receptor (CAR), a therapeutic composition, and at least one miRNA. In some embodiments, the engineered NPEV is derived from a parent CAR-induced pluripotent stem cell (iPSC). In some embodiments, the engineered NPEV does not express Programmed Cell Death Protein 1 (PD-1). In some embodiments, the engineered NPEV has increased anti-cancer or -tumor effects compared to the parent CAR-iPSC. In some embodiments, the engineered NPEV has lower toxicity compared to the parent CAR-iPSC. In some embodiments, the at least one miRNA comprises at least one miRNA which targets Lamin B2 (LMNB2). In some embodiments, the at least one miRNA comprises at least one miRNA which targets Programmed Cell Death Ligand 1 (PD-L1). In some embodiments, the engineered NPEV comprises dual miRNAs. In some embodiments, the engineered NPEV further comprises at least one additional miRNA. In some embodiments, the therapeutic composition is Chlorotoxin (CLTX).

In another aspect, the present invention provides a method of reducing or inhibiting growth of a cancer or tumor in a subject comprising administering to the subject an engineered NPEV as described herein. In some embodiments, the cancer or tumor is Glioblastoma (GBM).

Further disclosed herein is a method of determining effectiveness of an anti-cancer composition, wherein the cancer cells express at least one targetable marker of cancer. In some embodiments, the at least one targetable marker of cancer is PD-1. In some embodiments, the at least one targetable marker of cancer is Lamin B Receptor (LBR)

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows a schematic illustration where Human iPSCs are genetically engineered by CRISPR-Cas9 with CAR, chlorotoxin (CLTX), a 36-amino acid glioblastoma-targeting peptide. The CAR-iPSCs are differentiated into neutrophils (NPs) in a scalable vertical wheel bioreactor. The conditioned media is collected, and extracellular vesicles (EVs) are isolated. The EVs are loaded with different miRNAs targeting PD-L1. The CAR-NPEVs are used to test in vitro cytotoxicity of glioblastoma cells by various assays. The best formulation of miRNA cargo in CAR-NPEVs are selected based on various assays.

FIGS. 2A-2D show the characterization of EVs from CAR-iPSC differentiated neutrophils.

FIG. 2A shows nanoparticle tracking analysis with size distribution (i) and zeta potential (ii) shown.

FIG. 2B shows transmission electron microscopy images (i) large; (ii) small. Scale bar: 45 nm.

FIG. 2C shows a western blot of exosomal markers.

FIG. 2D shows exosome yield normalized to media usage.

FIGS. 3A-3B show a vertical-Wheel bioreactor-generated EV cargo analysis by proteomics and the miRNA-sequencing.

FIG. 3A shows proteomics analysis; Gene Ontology (GO) analysis of top 400 proteins in hiPSC-EVs.

FIG. 3B shows miRNA-sequencing data with Total miRNA counts for hiPSC-EVs EVs and the parent cells (i), and Heatmap illustration of top DEGs in miRNAs among different conditions (ii).

FIG. 4 shows a schematic illustration of how human brain organoids are formed from human iPSCs from healthy donors and GBM patient donors using brain tissue patterning factors in vitro. The dual miRNA CAR-iPSC-NPEVs are added to the 3D human brain organoid bearing GBM. The organoids are characterized by cell proliferation, apoptosis and PD-L1 downregulation.

FIGS. 5A-5D show the characterization of brain-like spheroids derived from human iPSK3 cells.

FIG. 5A shows K+ and Na+ currents (day 50 Purmo-cells).

FIG. 5B shows spontaneous excitatory post-synaptic currents (day 30 Purmo-cells). Purmo activates sonic hedgehog (SHH) which is required for hindbrain patterning. Cyclo inhibits SHH and is required for forebrain patterning.

FIG. 5C shows cyclo-treated cells and Purmo-treated cell phenotype (day 35).

FIG. 5D shows RT-PCR analysis (day 35); * p<0.05.

FIGS. 6A-6C show in vitro cytotoxicity of CAR-Neu EVs on GBM cells.

FIG. 6A shows CAR-Neu EV uptake by GBM cells; scale bar: 20 μm. Staining includes PKH EV labeling and Hoechst 33342 labeling.

FIG. 6B shows relative MTT activity. D21: day 21.

FIG. 6C shows relative mRNA expression (n=3). CAR-Neu EVs were added at 1×10⁹ and 1×10¹⁰ EVs/mL for 48 h. * indicates p<0.05. ** p<0.01, *** p<0.001.

FIG. 7 shows a schematic illustration of the experimental design for evaluation of the toxicology and pharmacodynamics after the CAR-iPSC-NPEV injection. For GBM models, it is also important to show the ability of the EVs to cross BBB and to test uptake by the recipient cells. In addition to the immunotherapeutic benefits, the potential mechanisms for antitumor cytotoxicity of the CAR-iPSC-NPEVs are also revealed.

FIGS. 8A-8B show a schematic overview of experimentation.

FIG. 8A shows an overview of experimental procedures.

FIG. 8B shows an overview of the differentiation protocol of iPSC-derived neutrophils used herein. Reproduced from Chang et al. Cell Report, 2022.

FIGS. 9A-9F show Proteomic Analysis of CLTX-CAR Neu exosome cargo.

FIG. 9A shows differential expression of serum proteins and other contaminants in day 21 EVs vs. day 12 EVs.

FIG. 9B shows differential expression of exosome and neutrophil markers.

FIG. 9C shows volcano plot of all significantly differentially expressed proteins in in day 21 EVs vs. day 12 EVs (301 low in d21 vs. d12, 155 high in d21 vs. d12, 456 Total DEPs).

FIG. 9D shows a GO pathway analysis of differentially expressed proteins in day 21 EVs vs. day 12 EVs.

FIG. 9E shows Heatmap of differential protein expression in day 21 EVs vs. day 12 EVs.

FIG. 9F shows a Ridgeline diagram of enriched proteins in day 21 EVs vs. day 12 EVs.

FIGS. 10A-10E show miRNA Analysis of CLTX-CAR Neu Exosome Cargo.

FIG. 10A shows total miRNA counts for d12 and d21 EVs.

FIG. 10B shows a PCA plot of d12 and d21 EVs showing the clusters of EVs.

FIG. 10C shows Heatmap analysis of top DEGs among d12 and d21 EVs.

FIG. 10D shows a volcano plot of DEGs in d21 EVs vs. d12 EVs.

FIG. 10E shows KEGG pathway analysis of top 50 miRNAs of d21 EVs.

FIGS. 11A-11C show cancer-Related Pathway Analysis of miRNAs.

FIG. 11A shows a Venn Diagram of DEGs in d12 and d21 EVs and genes associated with pathways in cancer.

FIG. 11B shows a Venn Diagram of DEGs in d12 and d21 EVs and genes associated with pathways in glioblastoma.

FIG. 11C shows a list of DEGS associated in pathways in glioblastoma differentially expressed in d21 EVs vs. d12 EVs.

FIGS. 12A-12C show Functional Prediction of miRNA cargo in the CAR-EVs.

FIG. 12A shows a flowchart of analysis.

FIG. 12B shows protein targets of d21 EV miRNAs (i) and functional prediction based on targets (ii).

FIG. 12C shows Protein targets of d12 EV miRNAs (i) and functional prediction based on targets (ii).

FIGS. 13A-13E show in Vitro Analysis of CAR-EVs in 2D GBM models.

FIG. 13A shows EV uptake assessed via fluorescence imaging for untreated cells (i), cells treated with a low dose of EVs (ii) and a high dose of EVs (iii-iv).

FIG. 13B shows histograms (i-iii) and total uptake (iv) of labelled EVs assessed via flow cytometry.

FIG. 13C-13E show in Vitro Analysis of CAR-EVs in 2D GBM models.

FIGS. 14A-14C show in Vitro Analysis of CAR-EVs in 3D GBM models.

FIG. 14A shows bright field images of tumor growth over the duration of 3D EV cytotoxicity assessment.

FIG. 14B shows size quantification of cell aggregates over the duration of experimentation.

FIG. 14C shows aggregate viability via trypan blue cell counting.

FIG. 15 shows Top DEPs based on proteomics analysis.

FIG. 16 shows Top 25 miRs based on miRNA-seq.

### DETAILED DESCRIPTION OF THE INVENTION

### Definitions

In this specification and in the claims which follow, reference will be made to a number of terms which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise.

The term “about” as used herein refers to +10% of a particular value. For example, “about 10”, as defined herein, encompasses any value between 9 and 11, including but not limited to 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “anticancer” and “anticarcinogen” refer to a substance, composition, or formula that counteracts the effects or inhibits the development of a cancerous cells and tissues. Anti-cancer agents encompass biotherapeutic anti-cancer agents as well as chemotherapeutic agents.

The “blood-brain barrier” or the “BBB” refers to the highly selective semipermeable border of endothelial cells that prevents certain small molecules circulating in the blood from crossing into the extracellular fluid of the central nervous system.

The term “cancer” is used to address any neoplastic disease and is not limited to epithelial neoplasms (surface and glandular cancers; such a squamous cancers or adenomas)). It is used here to describe both solid tumors and hematologic malignancies, including epithelial (surface and glandular) cancers, soft tissue and bone sarcomas, angiomas, mesothelioma, melanoma, lymphomas, leukemias and myeloma.

A “chimeric antigen receptor” (CAR) is an artificial T cell receptor used for immunotherapy. CAR are protein receptors that have been engineered to give T cells an enhanced ability to target a specific protein. CAR receptors are chimeric because the antigen binding and T cell activating functions have been combined into a single receptor.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., cancer). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises an engineered neutrophil extracellular vesicle (NPEV) comprising a cancer/tumor cell-targeting Chimeric antigen receptor (CAR), a therapeutic composition, and; at least one miRNA.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “cytotoxic” as used herein refers to the ability to kill a target cell. A cytotoxic T cell or NK cell may kill a target cell via target cell apoptosis using one or more different mechanisms including release of one or more cytotoxins or expression of a Fas ligand. In some embodiments, a cytotoxic T cell or NK cell kills a tumor cell via the release of one or more cytotoxins. “Cytotoxin” includes, but is not limited to, a perforin, a granzyme and a granulysin. Currently known granzymes are Granzyme A (GZMA), Granzyme B (GZMB), Granzyme H (GZMH), Granzyme K (GZMK), and Granzyme M (GZMM).

As used herein, cytotoxicity refers to the quality of being toxic to cells. Treating cells with a cytotoxic compound can result in a variety of cell fates, including necrosis (in which the cell membrane becomes compromised leading to cell lysis), senescence (in which the cell stops actively growing and dividing), or apoptosis (in which the cell activates a genetic program of controlled cell death).

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “detect” or “detecting” refers to an output signal released for the purpose of sensing of physical phenomenon. An event or change in environment is sensed and signal output released in the form of light.

As used herein, “diagnose”, “diagnosed,” “diagnosing”, and any grammatical variations thereof as used herein, refers to the act of process of identifying the nature of an illness, disease, disorder, or condition in a subject by examination or monitoring of symptoms.

As used herein, the term “genetically modified” refers to a living cell, tissue, or organism whose genetic material has been altered using genetic engineering techniques. The genetical modification results in an alteration that does not occur naturally by mating and/or natural recombination. Modified genes can be transferred within the same species, across species (creating transgenic organisms), and across kingdoms. New, exogenous genes can be introduced, or endogenous genes can be enhanced, altered, or knocked out.

The terms “inhibit,” “inhibiting,” and “inhibition” as used herein refer to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels.

The terms “immunotherapy” and “immunotherapeutic” refer to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

A “nucleic acid” is a chemical compound that serves as the primary information-carrying molecules in cells and makes up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

A “protein,” “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

By “reduce” or other forms of the word, such as “reducing” or “reduction,” is meant lowering of an event or characteristic (e.g., cancer/tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

The term “screening” refers to a method especially used in drug discovery in which data processing/control software, liquid handling devices, and sensitive detectors can allow for quick conductions of chemical, genetic, or pharmacological tests. This process allows one to quickly recognize active compounds, antibodies, or genes that modulate a particular biomolecular pathway. The results of these processes provide starting points for drug design.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “T cell” refers to a type of lymphocyte that is one of the most important white blood cells of the immune system. T cells can be distinguished from other lymphocytes by the presence of a T-cell receptor (TCR) on their cell surface. The immune-mediated cell death function of T cells is carried by two major subtypes: CD8⁺ “killer” T cells and CD4+ “helper T cells.

The term “therapeutically effective” refers to the amount of the composition used is of sufficient quantity to ameliorate one or more causes or symptoms of a disease or disorder. Such amelioration only requires a reduction or alteration, not necessarily elimination.

A “therapeutic composition” refers to at least one substance, molecule, or compound suitable for administering to a subject, wherein the composition further includes a pharmaceutical carrier. A non-limiting example includes a therapeutic composition comprises an exosome loaded with a chemotherapeutic agent, a small molecule inhibitor, an immunotherapeutic agent or a natural or designed anti-cancer toxin.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### General Description of Invention

Neutrophils (NPs) are a type of white blood cell (leukocytes) that act as the human innate immune system's first line of defense during the beginning (acute) phase of inflammation, particularly as a result of bacterial infection, environmental exposure, and some cancers. Neutrophils are a subset of granulocytes, a type of white blood cell. When stained with hematoxylin and eosin, neutrophils are observed to contain a nucleus divided into 2-5 lobes. They are formed from stem cells in the bone marrow and differentiate into subpopulations of neutrophil-killers and neutrophil-cagers. They are short-lived (between 5 and 135 hours) and highly mobile, as they can enter parts of tissue where other cells/molecules cannot.

Extracellular vesicles (EVs) are membrane-derived vesicles enclosed by phospholipid bilayers. EVs include vesicles of varied sizes from the smallest exosomes to the largest apoptotic bodies. EVs express various receptor and ligand molecules from source cells and are capable of interacting with target cells through these molecules. As EVs harbor various cargos such as proteins, mRNAs, and miRNAs derived from source cells, EVs are considered to be important mediators of intercellular communications. EVs bear functional and structural resemblance to synthetic drug carriers similar to liposomes, exosomes can be further used for drug delivery.

Recently, EVs have emerged as a novel drug delivery vehicle with high biological blood-brain barrier penetrability in GBM therapy. In particular, EVs can be engineered to increase target specificity and loaded with antitumor cargo (e.g., miRNAs) to mediate interconnected pathways in GBM.

Neutrophils also generate EVs in response to immunological stimuli during the inflammatory process. Neutrophil-derived EVs (NPEVs) are also known as ectosomes, microparticles, or microvesicles. These NPEVs share common features of general EVs, such as physical characteristics, membrane components, and mechanism of generation. There are several types of NPEVs; the classical neutrophil-derived microvesicles (NDMVs) and the alternative neutrophil-derived trails (NDTRs).

NDMVs are generated from neutrophils which arrive at the inflammatory foci. Membrane blebbing occurs in response to various stimuli at the inflammatory foci, and small parts of the blebs are detached from the neutrophils as NDMVs. On the other hand, NDTRs are generated from migrating neutrophils. The uropods of neutrophils are elongated by adhesion to endothelial cells, and small parts of the uropods are detached, leaving submicrometer-sized NDTRs. These 2 subtypes of neutrophil-derived EVs share common features such as membrane components, receptors, and ligands. However, there are substantial differences between these 2 neutrophil-derived EVs. NDTRs exert pro-inflammatory functions by guiding subsequent immune cells through the inflammatory foci. On the other hand, NDMVs exert anti-inflammatory functions by limiting the excessive immune responses of nearby cells.

The current invention discloses a NPEV loaded with an engineered miRNA and/or therapeutic composition as cargo and its use in reducing cancer/tumor load. In some embodiments, the cancer/tumor is GBM. Also disclosed are methods of using these NPEVs.

### Compositions

In one aspect, provided herein is an engineered neutrophil extracellular vesicle (NPEV) comprising a cancer/tumor-targeting chimeric antigen receptor (CAR), a therapeutic composition, and at least one miRNA.

Due to their short-life, induced pluripotent stem cells (iPSCs) bearing a CAR are an excellent source to generate neutrophils. Some examples of CARs, include, but are not limited to a CAR targeting CD19, B cell maturation antigen (BCMA), CD30, CD22, CD33, CD38, CD123, FLT3, NCAM1, CD5, CD70, MET, Muc1, L1CAM, CD44 SLAMF7, EGFR, EPHA2, GPC3, HER2, mesothelin, or PDCD1. In some embodiments, EVs secreted by CAR-iPSC-neutrophils or NPEVs express the CAR of the parent iPSC cell.

In one aspect, it is disclosed herein that delivery of miRNAs regulating PD-L1 and LMNB2 with CAR-iPSC-neutrophil secreted EVs significantly reduce cancer or tumor burden.

The miRNAs are small, single-stranded, non-coding RNA molecules. Found in plants, animals and some viruses, miRNAs are involved in RNA silencing and post-transcriptional regulation of gene expression. The miRNAs base-pair to complementary sequences in mRNA molecules, then silence and/or destabilize the said mRNA molecules by one or more of the following processes: (1) Cleavage of the mRNA strand into two pieces, (2) Destabilization of the mRNA by shortening its poly (A) tail, or (3) Reducing translation of the mRNA into proteins.

Some examples of tumor suppressor miRNAs include, but are not limited to, miR-1, miR-7, let-7, miR-9, miR-15a, miR-16 family, miR-18a, miR-25, miR-27a, miR-29 family, miR-30b, miR-31, miR-33 family, miR-34 family, miR-124, miR-126, miR-128, miR-145, miR-193b, miR-198, miR-204, miR-205, miR-206, miR-302, miR-335, miR-383, miR-449, miR-493, miR-504, miR-545 or miR-596. In some embodiments disclosed herein, the at least one miRNA target Lamin B2 (LMNB2) and/or Programmed Cell Death Ligand 1 (PD-L1).

In some embodiments, the at least one miRNA comprises two or more miRNAs. In some embodiments, the at least one miRNA comprises dual miRNAs. In some embodiments, the at least one miRNA comprises at least one miRNA targeting Lamin B2 (LMNB2). In some embodiments, the at least one miRNA is selected from miR-3148, miR-4698, miR-3133, miR-5700, or a combination thereof. In some embodiments, the at least one miRNA comprises miR-3133. In some embodiments, the at least one miRNA comprises at least one miRNA targeting Programmed Cell Death Ligand 1 (PD-L1). In some embodiments, the at least one miRNA is selected from miR-3117, miR-5193, miR-4282, miR-548, or a combination thereof. In some embodiments, the at least one miRNA comprises miR-5193.

In some embodiments, the engineered NPEV can comprise dual miRNAs and can further comprise at least one additional miRNA.

In some embodiments, the at least one additional miRNA selected from miR-34a, miR-424, miR-7, miR-10b, miR-20a, miR-21, miR-22, miR-25, miR-26a, miR-30a, miR-30c, miR-34c, miR-92a, miR-101, miR-103a, miR-125a, miR-143, miR-145, miR-146a, miR-148b, miR-181a, miR-182, miR-183, miR-191, miR-199a, miR-203a, miR-302b, miR-375, miR-378a, or a combination thereof.

In some embodiments, the at least one additional miRNA comprises miR-34a. In some embodiments, the at least one additional miRNA comprises miR-424.

In some embodiments, the engineered NPEV are enriched of one or more proteins selected from MDH2, AKRIB1, SERPINB9, RNPEP, EPCAM, NUTF2, PREP, CLDN6, THOP1, ENO2, or a combination thereof. In some embodiments, the enrichment of the engineered NPEV is measured in engineered NPEV isolated after 21 days of NP differentiation of CAR-iPSCs relative to an engineered NPEV isolated after 12 days of NP differentiation of CAR-iPSCs. In some embodiments, the engineered NPEV are de-enriched of one or more proteins selected from MAGED2, MATR3, CAD, THBS1, FUS, PRPF6, SMC4, PELP1, PSMD11, and TAF15. In some embodiments, the de-enrichment of the engineered NPEV is measured in engineered NPEV isolated after 21 days of NP differentiation of CAR-iPSCs relative to an engineered NPEV isolated after 12 days of NP differentiation of CAR-iPSCs.

In some embodiments, the CAR-iPSC-neutrophil can be further loaded with a therapeutic composition. The therapeutic composition can include but is not limited to a biotherapeutic agent, a chemotherapeutic agent, a small molecule inhibitor, an immunotherapeutic agent or a natural or designed anti-cancer toxin.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

Some examples of small molecule inhibitors of cancer include but are not limited to Imatinib (Gleevec) (BCR-ABL, PDGFR, SCF, KIT Inhibitor), Dasatinib (Sprycel) (BCR-ABL, SRC family (SRC, LCK, YES, FYN), and KIT, EPHA2, PDGFRB Inhibitor), Nilotinib (Tasigna) (BCR-ABL, PDGFRB, KIT Inhibitor), Bosutinib (Bosulif) (BCR-ABL, SRC-family (SRC, LYN, and HCK) Inhibitor), Ponatinib (Iclusig) (BCR-ABL, BCR-ABL (T315I), VEGFR, PDGFR, FGFR, EPH receptors, SRC families of kinases, KIT, RET, TIE2, FLT3 Inhibitor), Asciminib (Scemblix) (BCR-ABL, BCR-ABL (T315I) Inhibitor), Ripretinib (Quinlock) (KIT, PDGFRA, PDGFRA mutations, PDGFRB, TIE2, VEGFR2, BRAF Inhibitor), Avapritinib (Ayvakit) (KIT, KIT D816V, KIT exon 11, 11/17, and 17 mutants, PDGFRA and PDGFRA D842 mutants, PDGFRB, and CSFR1 Inhibitor), Gefitinib (Iressa) (EGFR and HER family Inhibitor), Erlotinib (Tarceva) (EGFR and HER family Inhibitor), Afatinib (Gilotrif) (EGFR and HER family Inhibitor), Osimertinib (Tagrisso) (EGFR and HER family Inhibitor), Dacomitinib (Vizimpro) (EGFR and HER family Inhibitor), Mobocertinib (Exkivity) (EGFR and HER family Inhibitor), Lapatinib (Tykerb) (EGFR and HER family Inhibitor), Neratinib (Nerlynx) (EGFR and HER family Inhibitor), Tucatinib (Tukysa) (EGFR and HER family Inhibitor), Osimertinib (Tagrisso) (EGFR and HER family Inhibitor), Crizotinib (Xalkori) (ALK, HGFR, c-Met, ROS1, RON), Ceritinib (Zykadia) (ALK, IGF-IR, InsR, ROS1), Alectinib (Alecensa) (ALK, RET), Brigatinib (Alunbrig) (ALK, ROS1, IGF-IR, FLT-3, EGFR deletion and point mutations inhibitors), Lorlatinib (Lorviqua) (ALK, ROS1, TYK1, FER, FPS, TRKA, TRKB, TRKC, FAK, FAK2, ACK inhibitor), Capmatinib (Tabrecta) (MET, MET exon 14 skipping inhibitor), Tepotinib (Tepmetko) (MET, MET exon 14 skipping inhibitor), Pralsetinib (Gavreto) (wild-type RET, oncogenic RET fusions (CCDC6-RET), RET mutations (RET V804L, RET V804M and RET M918T) inhibitor), Selpercatinib (Retevmo) (wild-type RET, multiple mutated RET isoforms inhibitor), Erdafitinib (Balversa) (FGFR1, FGFR2, FGFR3, FGFR4, RET, CSFIR, PDGFRA, PDGFRB, FLT4, KIT, VEGFR2 inhibitor), Pemigatinib (Pemazyre) (FGFR1, FGFR2, FGFR3 inhibitor), Infigratinib (Truseltiq) (FGFR1, FGFR2, FGFR3, FGFR4 inhibitor), Larotrectinib (Vitrakvi) (NTRK1, NTRK2, NTRK3 inhibitor), Entrectinib (Rozlytrek) (NTRK1, NTRK2, NTRK3, ROS1, ALK, JAK2, TNK2 inhibitor), Midostaurin (Rydapt) (FLT3 inhibitor), Gilteritinib (Xospata) (FLT3 inhibitor), Pexidartinib (Turalio) (CSFIR, KIT, FLT3 with ITD mutation Inhibitor), Vemurafenib (Zelboraf) (mutated forms of BRAF, wild-type BRAFCRAF, ARAF, SRMS, ACK1, MAP4K5, FGR inhibitor), Dabrafenib (Tafinlar) (BRAF V600E, BRAF V600K, and BRAF V600D, wild-type BRAF, CRAF, SIKI, NEK11, LIMK1 inhibitor), Encorafenib (Braftovi) (BRAF V600E, wild-type BRAF, CRAF, JNK1, JNK2, JNK3, LIMK1, LIMK2, MEK4, STK36 inhibitor), Trametinib (Mekinist) (MEK1, MEK2 Inhibitor), Cobimetinib (Cotellic) (MEK1, MEK2 Inhibitor), Binimetinib (Mektovi) (MEK1, MEK2 Inhibitor), Selumetinib (Koselugo) (MEK1, MEK2 Inhibitor), Idelalisib (Zydelig) (PI3Kδ inhibitor), Copanlisib (Aliqopa) (PI3Kα, PI3Kδ Inhibitor), Duvelisib (Copiktra) ((PI3Kα, PI3Kδ Inhibitor), Alpelisib (Piqray) (PI3Kα inhibitor), Umbralisib (Ukoniq) (PI3Kδ, CK1ε inhibitor), Ruxolitinib (Jakafi) (JAK1, JAK2 inhibitor), Fedratinib (Impact) (JAK2 inhibitor), Palbociclib (Ibrance) (CDK4, CDK6 Inhibitor), Ribociclib (Kisqali) (CDK4, CDK6 Inhibitor), Abemaciclib (Verzenio) (CDK4, CDK6 Inhibitor), Trilaciclib (Cosela) (CDK4, CDK6 Inhibitor), Ibrutinib (Imbruvica) (BTK Inhibitor), Acalabrutinib (Calquence) (BTK Inhibitor), Zanubrutinib (Brukinsa) (BTK Inhibitor), Enasidenib (Idhifa) (IDH1 and IDH2), Ivosidenib (Tibsovo) (IDH1 and IDH2 Inhibitor), Tirbanibulin (Klisyri) (SRC Inhibitor).

Some examples of immunomodulatory agents include but are not limited to Amivantamab, Cetuximab, Nimotuzumab, Panitumumab, Bevacizumab, Ramucirumab, Nivolumab, Pembrolizumab, Cemiplimab, Atezolizumab, Avelumab, Durvalumab, Ertumaxomab, Margetuximab, Pertuzumab, Trastuzumab, Trastuzumab duocarmazine, Trastuzumab emtansine, Rituximab, Human or humanized anti-CD20 antibodies, Ibritumomab, Brentuximab (+ mono methyl auristatin E), Alemtuzumab, and Ipilimumab (Yervoy).

Exemplary natural or designed anti-cancer agents include but are not limited to Pseudomonas exotoxin A (PE, ETA), Diphtheria toxin (DT), Ricin, Shiga toxin (Stx), Abrin, Barnase, Binase, Anthrax toxin, KillerRed, miniSOG, Granzyme B, Botulinum neurotoxin, Listeriolysin O or Streptolysin-O.

In some embodiments the therapeutic composition is chlorotoxin (CLTX), a 36-amino acid GBM-targeting peptide (PubChem CID 86278273). CLTX as used herein can be alternatively referred to as “TM601 peptide,” “UNII-06UV5RFW57,” “TM 601,” or “TM-601”. CLTX is a 36 amino acid peptide neurotoxin found in the venom of the deathstalker scorpion with anticancer property. CLTX is a chloride channel blocker and preferentially binds to glioma cancer cells via the transmembrane endopeptidase matrix metalloproteinase-2 (MMP-2), thereby preventing the spread of tumor cells. In some embodiments, CLTX comprises the amino acid sequence of MCMPCFTTDHQMARKCDDCCGGKGRGKCYGPQCLCR (SEQ ID NO: 1), or an amino acid at least about 70%, at least about 75%, at least about 80%, at least about 85%, at least about 90%, at least about 95%, at least about 96%, at least about 97%, at least about 98%, at least about 99%, or more, identical thereto. In some embodiments, the CLTX comprises the amino acid sequence of MCMPCFTTDHQMARKCDDCCGGKGRGKCYGPQCLCR (SEQ ID NO: 1), comprising disulfide bonds between Cys2-Cys19, Cys5-Cys-28, Cys16-Cys33, Cys20-Cys35, or a combination thereof. In some embodiments, CLTX is represented in the image below:

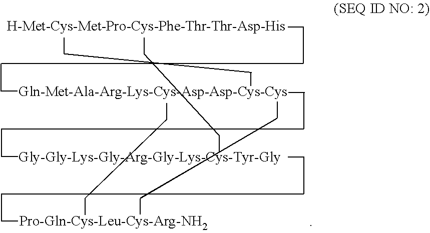

In some embodiments, the engineered NPEV has a size of between about 30 nanometers and about 200 nanometers, between about 40 nanometers and 180 nanometers, between about 30 nanometers and 150 nanometers, between about 25 nanometers and about 100 nanometers, between about 100 nanometers and 200 nanometers, or between about 75 nanometers and 150 nanometers. In some embodiments, the engineered NPEV has a size of about 115 nanometers.

In some embodiments, the engineered NPEV has a zeta potential of between about −10 millivolt and about −40 millivolt, between about −15 millivolt and −25 millivolt, between about −10 millivolt and about −20 millivolt, between about −20 millivolt and about −35 millivolt. In some embodiments, the engineered NPEV has a zeta potential of about −20 millivolt, about −21 millivolt, about −22 millivolt, about −23 millivolt, about −24 millivolt, about −25 millivolt, about −26 millivolt, about −27 millivolt, about −28 millivolt, about −29 millivolt, about −30 millivolt, about −31 millivolt, about −32 millivolt, about −33 millivolt, about −34 millivolt, or about −35 millivolt. In some embodiments, the engineered NPEV has a zeta potential of about −29.68 millivolt.

In an alternative aspect, provided herein is a pharmaceutical composition comprising an engineered NPEV as described herein, and a pharmaceutically acceptable carrier. In some embodiments, the engineered NPEV is formulated in a dose of between about 1×10⁷ EVs/mL and about 1×10¹¹ EVs/mL. In some embodiments, the engineered NPEV is formulated in a dose of 0.01×10⁹ EVs/mL, 0.1×10⁹ EVs/mL, about 1×10⁹ EVs/mL, or about 1×10¹⁰ EVs/mL.

In some embodiments, the engineered NPEV is administered to a subject in need thereof by intravenous injection, intratumoral injection, or a combination thereof.

### Methods of Treatment

In another aspect, the present invention provides a method of reducing or inhibiting growth of a cancer/tumor by administering the engineered NPEV described above to a subject positive for the cancer/tumor.

In some embodiments, a subject is first tested to detect cancer/tumor cell markers. Some exemplary tests to detect cancer/tumor cell markers in a subject include but are not limited toa blood chemistry test, complete blood count (CBC), cytogenetic analysis, immunophenotyping, liquid biopsy, sputum cytology, tumor marker tests, urinalysis, urine cytology, imaging tests used in cancer, CT scan, MRI, nuclear scan, bone scan, PET scan, ultrasound, X-rays, or biopsy.

In some embodiments, the method of reducing or inhibiting growth of a cancer or tumor comprises administering to a subject positive for a cancer or tumor marker including but not limited to ALK gene rearrangements and overexpression, Alpha-fetoprotein (AFP), B-cell immunoglobulin gene rearrangement, BCL2 gene rearrangement, BCR-ABL fusion gene (Philadelphia chromosome), Beta-2-microglobulin (B2M), Beta-human chorionic gonadotropin (Beta-hCG), Bladder Tumor Antigen (BTA), BRAF V600 mutations, BRCA1 and BRCA2 gene mutations, CA15-3/CA27.29, CA19-9, CA-125, CA 27.29, Calcitonin, Carcinoembryonic antigen (CEA), CD19, CD20, CD22, CD25, CD30, CD33, CD31, FL1, CD34, Chromogranin A (CgA), Chromosome 17p deletion, Chromosomes 3, 7, 17, and 9p21, Circulating tumor cells of epithelial origin, C-kit/CD117, Cyclin D1 (CCND1) gene rearrangement or expression, Cytokeratin fragment 21-1, Calretinin, CD34, CD99, CD117, Desmin, Des-gamma-carboxy prothrombin (DCP), DPD gene mutation, Epithelial membrane antigen (EMA), EGFR, ESRI gene mutation, Estrogen receptor (ER)/progesterone receptor (PR), FGFR2 and FGFR3 gene mutations, Factor VIII, Fibrin/fibrinogen, 5-HIAA, 5-Protein signature (OVA1), FLT3 gene mutations, 46-Gene signature (Prolaris), FoundationOne CDx (F1CDx) genomic test, FoundationOne Liquid CDx, Gastrin, Glial fibrillary acidic protein (GFAP), Gross cystic disease fluid protein (GCDFP-15), Guardant360 CDx genomic test, HMB-45, Human chorionic gonadotropin (hCG), HE4, HER2/neu (ERBB2) gene amplification, mutations, protein overexpression, IDH1 and IDH2 gene mutations, Immunoglobulins, IRF4 gene rearrangement, JAK2 gene mutation, keratin (various types), KRAS gene mutation, Lactate dehydrogenase, lymphocyte marker (various types), MART-1 (Melan-A), Myo D1, muscle-specific actin (MSA), Microsatellite instability (MSI) and/or mismatch repair deficiency (dMMR), MYC gene expression, MYD88 gene mutation, Myeloperoxidase (MPO), Neuron-specific enolase (NSE), NTRK gene fusion, neurofilament, Nuclear matrix protein 22, PCA3 mRNA, PIK3CA gene mutation status, PML/RARa fusion gene, Programmed death ligand 1 (PD-L1), Prostate-specific antigen (PSA), placental alkaline phosphatase (PLAP), Prostatic Acid Phosphatase (PAP), RET gene fusions and mutations, ROS1 gene rearrangement, 17-Gene signature (Oncotype DX GPS test), 70-Gene signature (Mammaprint), Soluble mesothelin-related peptides (SMRP), Somatostatin receptor, S100 protein, smooth muscle actin (SMA), synaptophysin, T-cell receptor gene rearrangement, Terminal transferase (TdT), thymidine kinase, thyroglobulin (Tg), thyroid transcription factor-1 (TTF-1), Tumor M2-PK, Thiopurine S-methyltransferase (TPMT) enzyme activity or TPMT gene, Thyroglobulin, TP53 gene mutations, Tumor mutational burden (TMB), 21-Gene signature (Oncotype DX), UGT1A1*28 variant homozygosity, Urine catecholamines: VMA and HVA, Urokinase plasminogen activator (uPA) and plasminogen activator inhibitor (PAI-1), or vimentin.

In some embodiments, the engineered NPEV, or pharmaceutical composition comprising thereof, of any preceding aspect treats or prevents a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

The engineered neutrophil extracellular vesicle (NPEV) comprising a cancer/tumor-targeting chimeric antigen receptor (CAR), a therapeutic composition, and at least one miRNA may be administered in such amounts, time, and route deemed necessary in order to achieve the desired result. The exact amount of the engineered NPEV, or pharmaceutical composition comprising thereof, varies from subject to subject, depending on the species, age, and general condition of the subject, the severity of the cancer, the particular therapeutic composition, its mode of administration, its mode of activity, and the like. The engineered NPEV, or pharmaceutical composition comprising thereof, is preferably formulated in dosage unit form for ease of administration and uniformity of dosage. It is understood, however, that the total daily usage of the engineered NPEV, or pharmaceutical composition comprising thereof, will be decided by the attending physician within the scope of sound medical judgment. The specific therapeutically effective dose level for any particular subject will depend upon a variety of factors including the type of cancer, location of the cancer/tumor and malignancy being treated and the severity of the cancer/tumor; the activity of the engineered NPEV, or pharmaceutical composition comprising thereof, employed; the specific engineered NPEV, or pharmaceutical composition comprising thereof, employed; the age, body weight, general health, sex and diet of the patient; the time of administration, route of administration, and rate of excretion of the specific engineered NPEV, or pharmaceutical composition comprising thereof, employed; the duration of the treatment; drugs used in combination or coincidental with the specific engineered NPEV, or pharmaceutical composition comprising thereof, employed; and like factors well known in the medical arts.

The exact amount of engineered NPEV, or pharmaceutical composition comprising thereof, required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

The engineered NPEV, or pharmaceutical composition comprising thereof, may be administered by any route. In some embodiments, the engineered NPEV, or pharmaceutical composition comprising thereof, is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the engineered NPEV, or pharmaceutical composition comprising thereof (e.g., its stability in the environment of the gastrointestinal tract), the condition of the subject (e.g., whether the subject is able to tolerate oral administration), etc.

In some embodiments, the engineered NPEV, or pharmaceutical composition comprising thereof, is administered to the subject 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the engineered NPEV, or pharmaceutical composition comprising thereof, is administered daily. In some embodiments, the engineered NPEV, or pharmaceutical composition comprising thereof, is administered every day, every 2 days, every 3 days, every 4 days, every 5 days, every 6 days, every 7 days, or more. In some embodiments, the engineered NPEV, or pharmaceutical composition comprising thereof, is administered every week, every 2 weeks, every 3 weeks, every 4 weeks, or more. In some embodiments, the engineered NPEV, or pharmaceutical composition comprising thereof, is administered every month, every 2 months, every 3 months, every 4 months, every 5 months, every 6 months, every 7 months, every 8 months, every 9 months, every 10 months, every 11 months, every 12 months, or more. In some embodiments, the engineered NPEV, or pharmaceutical composition comprising thereof, is administered every year, every 2 years, every 3 years, every 4 years, every 5 years, or more.

The concentration of active agent(s) can vary widely and are selected primarily based on activity of the active ingredient(s), body weight and the like in accordance with the particular mode of administration selected and the patient's needs. Concentrations, however, can typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or prophylactic regimen in a particular subject or group of subjects.

In some embodiments, the NPEV-CAR further comprises a therapeutic composition, and at least one miRNA can be prepared as a “concentrate.” In some embodiments, the at least one miRNA is prepared as a concentrate in a storage container of a premeasured volume and/or a predetermined amount ready for dilution. In some embodiments, the at least one miRNA is prepared as a concentrate in a soluble capsule ready for addition to a specified volume of water, saline, alcohol, hydrogen peroxide, or other diluent.

In some embodiments, the method of reducing or inhibiting growth of a cancer/tumor comprises a cancer of any preceding aspect. Subjects who test positive for any one of the above-mentioned cancers can be treated with an engineered NPEV as described herein, wherein the NPEV comprises a cancer/tumor-targeting CAR, a therapeutic composition, and at least one miRNA of any preceding aspect.

In some embodiments, the cancer or tumor is Glioblastoma. Some therapeutic compositions to inhibit or reduce the growth of glioblastoma include but are not limited to chemotherapeutic, small molecule inhibitor or immunotherapeutic treatments which further include but are not limited to Temozolomide, Carmustine (BCNU), Lomustine (CCNU), Fotemustine, Bevacizumab, Irinotecan (CPT-11), Veliparib (ABT-888), Olaparib (AZD-2281, MK-7339), Niraparib (MK-4827), Pamiparib (BGB-290), Cediranib (AZD-2171), Gossypol (AT-101), Cabozantinimb (XL-184), Erlotinib, Gefitinib, mafodotin (ABT-414), Depatuxizumab, Imatinib, Dasatinib, Sorafenib, Sunitinib, Temsirolimus (CCI-779), Everolimus, Cemiplimab, Nivolumab, Rindopepimut peptide vaccine, DCVax®-L or VB-111 (Ofranergene obadenovec) gene therapy using an adenovirus type 5 vector. Some examples of miRNAs that inhibit different cell signaling networks, transcription factors, and anti-apoptotic genes to inhibit glioblastoma growth include but are not limited to miR-302-367, miR-Cdh4, miR-378a-3p, miR-342, miR-153, miR-940, miR-7-5P, miR-101, and miR-338.

Reducing or inhibiting growth of a cancer or a tumor comprises a decrease in the size, proliferation, differentiation, metastasis, and/or activity of the cancer or the tumor in a subject. The decrease can be a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to a standard or control.

### Screening Methods

In an alternative aspect, provided herein is a method of determining effectiveness of an anti-cancer composition, using an engineered NPEV as described herein comprising a cancer/tumor-targeting CAR, a therapeutic composition, and at least one miRNA. This is a method of screening a therapeutic composition, CAR, or a miRNA of any preceding aspects that can be used to inhibit or reduce the growth of a cancer/tumor of any preceding aspect wherein the cancer/tumor expresses at least one targetable marker of cancer, wherein the targetable marker can be of any preceding aspect. In some embodiments, the at least one targetable marker of cancer is PD-1. In some embodiments, the at least one targetable marker of cancer is Lamin B Receptor (LBR).

The method of determining effectiveness of an anti-cancer composition can be tested by evaluating the at least one engineered NPEV as described herein loaded with selected therapeutic compositions of any of the preceding aspects, in cancer cell lines and 3D human organoids bearing cancer/tumor in vitro. Cytotoxicity tests of at least one engineered NPEV as described herein can be performed in 3D cancer/tumor organoid models. The toxicological and pharmacodynamic evaluation of the at least one engineered NPEV as described herein can be performed in cancer/tumor models in vivo.

The first step to engineering an NPEV that can be used to treat or reduce cancer/tumor burden is to load CAR-iPSC-NPEVs with miRNAs and perform cytotoxicity evaluation in vitro. In some embodiments, miRNAs in CAR-iPSC-NPEVs (bio-manufactured in a Vertical Wheel bioreactor) are formulated using electroporation and screened in vitro. In some embodiments, the in vitro screening comprises proliferation and cytotoxicity studies against cancer/tumor cells. In some embodiments, thereafter, the engineered NPEV is loaded with a therapeutic composition as described in any of the preceding aspects.

Some exemplary cancer/tumor cell lines include but are not limited to NCI-H295R, 5637, HT-1376, J82, SW 780, T24T24-Luc-Neo, MG-6, Saos-2, SJSA-1, SW 1353, D54-Luc, DBTRG-05M, GGli36-DsRed-R-Luc (rescued), LN-18, LN-229, LN-827 LucNeo, M059K, SF-295, SF-539, SF-767, SNB-19, U-251, U-251-Luc-mCh-Puro, U-87 MG, U-87 MG-Luc, HeL, aKB, C2BBe1, Caco-2, COLO 205, COLO 205-Luc #2, DLD-1, HCC2998, HCT-116, HCT-116-Luc, HCT-15, HCT-8, HT-29, HT-29-Luc, LoVo, LS 174T, LS411N, NCI-H508, SW-480, SW-620, KLE, KLE-Luc-mCh-Puro, A-431, HEKn, HEKn-2, OE33, A-673, RD-ES, HT-1080, GIST-T1, NCI-N87, NUGC-4, MKN-45, NCI-N87, SNU-5, CAL 27, FaDu, KARPAS 299, HL-60, EOL-1, Kasumi-1, Kasumi-3, Kasumi-3-Luc-mCh-Puro, KG-1-Luc-mCh-Puro, MOLM-13, MV-4-11, MV-4-11-Luc-mCh-Puro, NOMO-1, THP-1, NALM6, NALM6-Luc-MCh-Puro, Reh, Reh Luc-Neo, RS4;11, K-562, K-562-Luc2, HEL, HEL 92.1.7, HEL 92.1.7-Luc-Neo, HEL-Luc-Neo, ARH-77, CCRF-CEM, DND-41-Luc-mCh-Puro, Jurkat, Jurkat-Clone E6-1, MOLT-4, MOLT-4-Luc-MCh-Puro, SW 872, Hep 3B2.1-7, Hep G2, Calu-6, HCC2935, NCI-H1703, NCI-H1703-Luc-mCh-Puro, NCI-H2030, NCI-H2110, NCI-H2122, NCI-H3122, NCI-H322, MNCI-H82, A549, A549-Luc-C8, Calu-1, Calu-3, HCC827, HCC827-Luc-mCh-Puro, NCI-H125, NCI-H125-Luc, NCI-H1299, NCI-H1650, NCI-H1975, NCI-H1975-Luc, NCI-H23, NCI-H292, NCI-H441, NCI-H460, NCI-H460-Luc2, NCI-H522, PC-9, PC-9-Luc-mCh-puro, DMS 114, NCI-H446, NCI-H69, SHP-77, EBC-1, SK-MES-1, DB, DBM2, RL, Farage, B-JAB, Daudi, Daudi-Luc-mCh-Puro, NAMALWA, Raji, Raji-Luc, Ramos, Ramos-Luc, HuT 78, HT, SU-DHL-6, SU-DHL-6-Luc-mCh-Puro, GRANTA-519, OCI-Ly1 LN, OCI-Ly1 R10-Luc-mCh-Puro, OCI-Ly1 R7-Luc-mCh-Puro, OCI-Ly19-Luc-Neo, OCI-Ly3-Luc-mCh-Puro, OCI-Ly7-Luc-Neo, Pfeiffer, SU-DHL-10, SU-DHL-10-LN-High, SU-DHL-16, SU-DHL-4-Luc-mCh-Puro, SU-DHL-8, TMD8, Toledo-Luc-Neo, WSU-DLCL2, WSU-FSCCL, NK-92 MI, KARPAS 299, HCC1395, HCC1806, AU-565, BT-20, BT-474, HCC70, Hs 578Bst, Hs 578T, MCF 10A, MCF-7, MCF7-Luc-mCh-Puro, MDA-MB-231, MDA-MB-231-2LMP, MDA-MB-231-D3H2LN, MDA-MB-231-Luc-D3H1, MDA-MB-231-Luc-D3H2LN, MDA-MB-361, MDA-MB-453, MDA-MB-468, MX-1-Luc, SK-BR-3, T47D, ZR-75-1, A2058, A375, COLO 829, G-361, LOX-IMVI, M14, MDA-MB-435S, OCM-1, OCM-1-Luc-mCh-Puro, SK-MEL-5, SK-MEL-28, SK-MEL-28-Luc-mCh-Puro, UACC-62, WM-115, WM-266-4, JJN-3-Luc, KMS-11, KMS-26, KMS-34, MM.1S (pMMP-Luc-Neo), NCI-H929, NCI-H929-Luc-mCh-Puro, OPM-2, RPMI 8226, U266B1, SK-N-AS, SK-N-FI, SK-N-SH, MKL-1, Hs 895.Sk, MRC-5, A2780, A2780-Luc, IGROV1, IGROV1-Luc-Mch-Puro, NIH: OVCAR-3, NIH: OVCAR-3-Luc-mCh-Puro, OV-90, OVCAR-4, OVCAR-5, OVCAR-5-Luc-mCh-Puro, OVCAR-8, OVCAR-8-Luc-mCh-Puro, PA-1, SK-OV-3 (Subcutaneous), SK-OV-3-Luc-D3 (Intraperitoneal), AsPC-1, Bx-PC-3, BxPC-3-Luc2, Capan-1, Capan-2, KP4, MIA PaCa-2, MIA PaCa-2-Luc, PANC-1, PANC-1-Luc, SU-86.86, SW 1990, Detroit 562, BeWo, 22Rv1, CWR-22-R, DU 145, DU 145-Luc, LnCap clone FGC, PC-3, PC-3-Luc, PC-3M-Luc-C6, VCaP, 293, 293-Luc-mCh-Puro, 769-P, 786-O, 786-O-Luc-Neo (rescued), A-498, ACHN, Caki-1, TK-10, RPTEC, MB-1, TT, SK-LMS-1.

In some embodiments, the engineered NPEVs are evaluated in 3D human organoid bearing tumor in vitro. In some embodiment, the organoid comprises a human brain organoid bearing GBM. Human brain organoids bearing GBM can be constructed with vascularization. In some embodiments, cytotoxicity tests of the engineered NPEVs as described herein can be performed in 3D GBM organoid models.

In vitro 3D cancer/tumor organoid models and in vivo models of any one of the above-mentioned cancers involving organs such as the brain, breast, colon, leukemia, liver, lung, lymphoma, myeloma, ovarian, pancreas, prostate, renal, can be used to perform cytotoxicity tests and to test the effectiveness of the at least one engineered NPEV as described herein.

Decrease in targetable markers, size, proliferation, differentiation, metastasis, and/or activity of the cancer/tumor in 3D organoids or in vivo indicates effectiveness of the at least one engineered NPEV as described herein loaded with a therapeutic composition. The decrease can be a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of decrease in between as compared to a standard or control.

Increase in cytotoxicity in cell lines, size, proliferation, differentiation, metastasis, and/or activity of the cancer/tumor in 3D organoids or in vivo indicates effectiveness of the at least one engineered NPEV as described herein loaded with a therapeutic composition. The increase can be a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of increase in between as compared to a standard or control.

In some embodiments, the toxicological and pharmacodynamic parameters of an engineered NPEV as described herein are evaluated in cancer/tumor models in vivo. In some embodiments, the engineered NPEVs can thereafter be used to reduce or inhibit the growth of a cancer/tumor.

### Embodiments

1. An engineered neutrophil extracellular vesicle (NPEV) comprising:
(a) a cancer- or tumor cell-targeting Chimeric antigen receptor (CAR);
(b) a therapeutic composition; and
(c) at least one miRNA.

2. The engineered NPEV of embodiment 1, wherein the engineered NPEV is derived from a parent CAR-induced pluripotent stem cell (iPSC).

3. The engineered NPEV of embodiment 1 or 2, wherein the engineered NPEV does not express Programmed Cell Death Protein 1 (PD-1).

4. The engineered NPEV of embodiment 2 or 3, having increased anti-cancer or anti-tumor effects compared to the parent CAR-iPSC.

5. The engineered NPEV of any one of embodiments 2-4, having lower toxicity compared to the parent CAR-iPSC.

6. The engineered NPEV of any one of embodiments 1-5, wherein the at least one miRNA comprises at least one miRNA targeting Lamin B2 (LMNB2).

7. The engineered NPEV of any one of embodiments 1-6, wherein the at least one miRNA is selected from miR-3148, miR-4698, miR-3133, miR-5700, or a combination thereof.

8. The engineered NPEV of any one of embodiments 1-7, wherein the at least one miRNA comprises miR-3133.

9. The engineered NPEV of any one of embodiments 1-8, wherein the at least one miRNA comprises at least one miRNA targeting Programmed Cell Death Ligand 1 (PD-L1).

10. The engineered NPEV of any one of embodiments 1-10, wherein the at least one miRNA is selected from miR-3117, miR-5193, miR-4282, miR-548, or a combination thereof.

11. The engineered NPEV of any one of embodiments 1-10, wherein the at least one miRNA comprises miR-5193.

12. The engineered NPEV of any one of embodiments 1-9, wherein the at least one miRNA comprises two or more miRNAs.

13. The engineered NPEV of any one of embodiments 1-12, wherein the at least one miRNA comprises dual miRNAs.

14. The engineered NPEV of any one of embodiments 1-13, further comprising at least one additional miRNA.

15. The engineered NPEV of any one of embodiments 1-14, further comprising at least one additional miRNA selected from miR-34a, miR-424, miR-7, miR-10b, miR-20a, miR-21, miR-22, miR-25, miR-26a, miR-30a, miR-30c, miR-34c, miR-92a, miR-101, miR-103a, miR-125a, miR-143, miR-145, miR-146a, miR-148b, miR-181a, miR-182, miR-183, miR-191, miR-199a, miR-203a, miR-302b, miR-375, miR-378a, or a combination thereof.

16. The engineered NPEV of any one of embodiments 1-16, wherein the therapeutic composition is Chlorotoxin (CLTX).

17. The engineered NPEV of any one of embodiments 1-16, wherein the NPEV has a size of between about 30 nanometers and about 200 nanometers.

18. The engineered NPEV of any one of embodiments 1-27, wherein the NPEV has a zeta potential of between about −10 millivolt and about −40 millivolt.

19. A pharmaceutical composition comprising the engineered NPEV of any one of embodiments 1-18, and a pharmaceutically acceptable carrier.

20. The pharmaceutical composition of embodiment 19, wherein the engineered NPEV is formulated in a dose of between about 1×10⁷ EVs/mL and about 1×10¹¹ EVs/mL.

21. A method of reducing or inhibiting growth of a cancer or a tumor in a subject, comprising administering to the subject an engineered NPEV of any one of embodiments 1-18 or a pharmaceutical composition of embodiment 19 or 20.

22. The method of embodiment 21, wherein the cancer or tumor is selected from non-small cell lung cancer (NSCLC), breast cancer, acute leukemia, Glioblastoma (GBM).

23. The method of embodiment 21 or 22, wherein the cancer or tumor is GBM.

24. The method of any one of embodiments 21-23, wherein the engineered NPEV is administered by intravenous injection, intratumoral injection, or a combination thereof.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. Neutrophil Derived Extracellular Vesicles for miRNA Delivery to Glioblastoma

In a first example, it is disclosed that CAR-iPSC-neutrophil secreted extracellular vesicles are used for miRNA delivery to glioblastoma.

Role of CAR-iPSC-Neutrophil Derived EVs in miRNA Delivery

Cell-secreted EVs, with a size of 30-200 nm and referred as “exosomes,” have emerged as promising systems to encapsulate various active biomolecules including nucleic acids, lipids and proteins. EVs are present in many body fluids and possess intrinsic transmembrane and membrane-anchored proteins to prolong blood circulation and promote tissue-directed delivery and cellular uptake of internal contents. It has been shown that natural killer (NK) cell-derived EVs (NK-EVs) carrying cargo such as cytotoxic proteins, miRNAs, and cytokines employ multiple mechanisms to kill tumor cells, but also exhibit immunomodulatory activity by stimulating other immune cells. The effects of CAR-neutrophils have been shown to be more potent than CAR-NK cells. CAR-Neutrophils have recently shown to have potent anti-GBM effects, but are limited by short life span, unpredictable adverse events akin to CAR-T including potential neurotoxicity, on-target off-tumor effects, cytokine release syndrome, while EVs have the benefit of reduced theoretical side effect profile, increased BBB penetration and similar/superior efficacy by interacting with tumor and/or immune cells and can also sense the actual immune cell status in cancer patients.

Neutrophil-EVs were also shown to regulate immune cell polarization, possibly by expressing various parent cell receptors and cytotoxic molecules, which can mediate cytotoxic activity against tumor cells. In this study, CAR-iPSC-NPEVs are prepared using scalable Vertical Wheel bioreactors by established procedures and miRNAs (targeting PD-L1 and LMNB2) are delivered as payloads which lead to enhanced preclinical outcomes, laying a foundation for future lead to Phase 1 human clinical studies.

### A. Innovation

Important aspects of this invention include but are not limited to:
1. The first study to formulate miRNAs targeting LMNB2 and PD-L1 using EVs obtained from CAR-iPSC-neutrophils in a Vertical Wheel bioreactor, which are fully characterized for their cargo using proteomics and miRNA-sequencing;
2. The first study to understand the role of CAR-neutrophil EVs containing miRNAs against glioblastoma;
3. Understanding of molecular mechanisms which are involved in overcoming this resistance uncover novel therapeutic targets in GBM; and
4. Access to a large biorepository of human and human-derived specimens (over 13,000 unique specimens). These have also enabled the establishment of dedicated bank of patient-derived glioma stem cell-enriched lines that grow in vitro and in vivo, which further increases the translational potential of the experiments proposed below.

### Approach:

Loading CAR-iPSC-NPEVs with PD-L1 and LMNB2 miRNAs and Perform Cytotoxicity Evaluation In Vitro.

CAR-iPSC-NPEVs loaded with PD-L1 and LMNB2 miRNAs reduces the cytotoxicity of human GBM cells in vitro.

### Purpose, Rationale and Design:

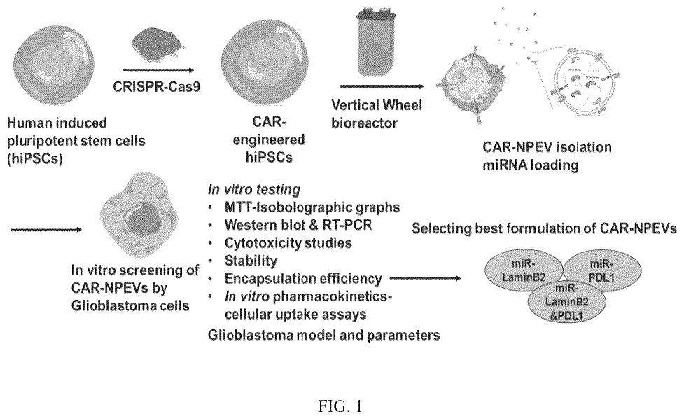

miRNAs screening using PD-L1 and LaminB2 as targets initially selected miR-5193 (PD-L1) and miR-3133 (LMNB2). These miRNAs (50 nM) downregulate PD-L1 and Lamin B2. Four miRNAs for LMNB2 and PD-L1 in CAR-iPSC-NPEV formulation are screened for their in vitro activity. The most effective formulation for each of the miRNA is further loaded to NPEVs. The CAR is targeting GBM with CLTX, a 36-amino acid peptide. The CLTX-T-CAR neutrophils have been shown to possess a typical neutrophil phenotype and killed the GBM cells through membrane-associated matrix metalloproteinase 2 (MMP2). In vitro cytotoxicity assay of the formulated CAR-iPSC-NPEVs is performed using GBM cells and GBM patient-derived stem-cell enriched cell lines provided by Mayo Clinic, Jacksonville, FL. as shown in FIG. 1.

### Studies:

### Isolation and Characterization of CAR-iPSC-Neutrophil Derived EVs (NPEVs):

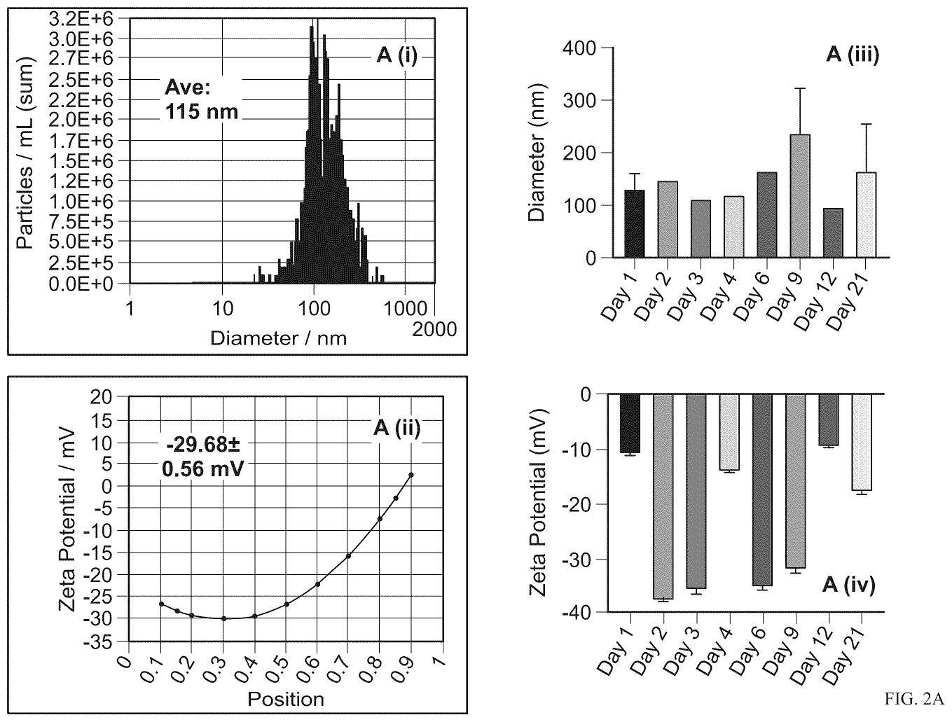

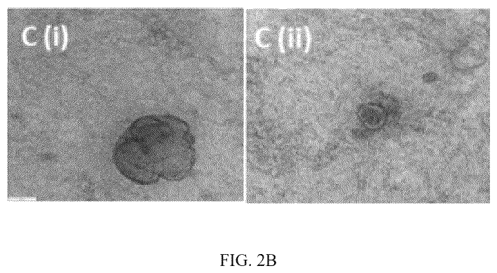

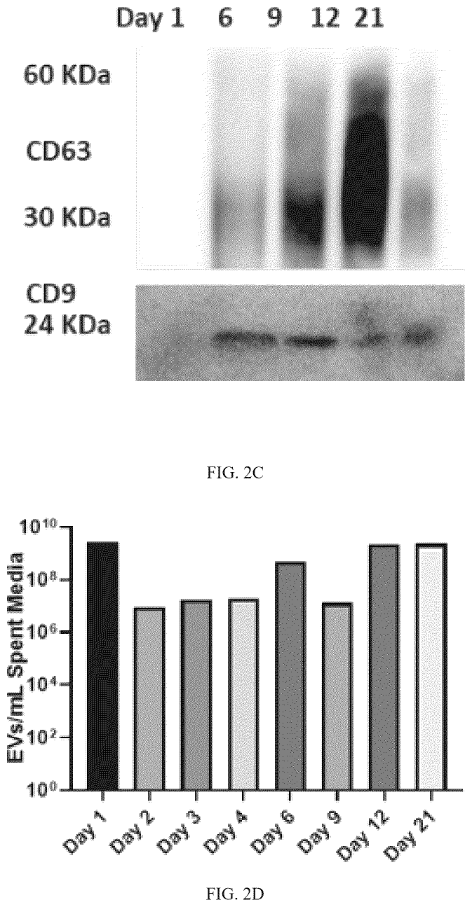

EVs have been isolated from the conditioned medium over the course of NP differentiation of CAR-iPSCs at day 1, 3, 6, 9, 12, and 21 (FIG. 2A-2D). Nanoparticle tracking analysis showed day 21 sample has an average size of 115 nm (in exosome size range of 30-200 nm) and zeta potential of −29.68 mV (FIG. 2A). Transmission electron microscopy shows the typical cup-shape exosome morphology (FIG. 2B). Western blot demonstrates expression of exosomal marker CD63 and CD9 (FIG. 2C). These results demonstrate the feasibility of isolating and characterizing CAR-iPSC-NPEVs.

Scaling Up EV Production in Vertical Wheel Bioreactors and Characterization with Multi-Omics:

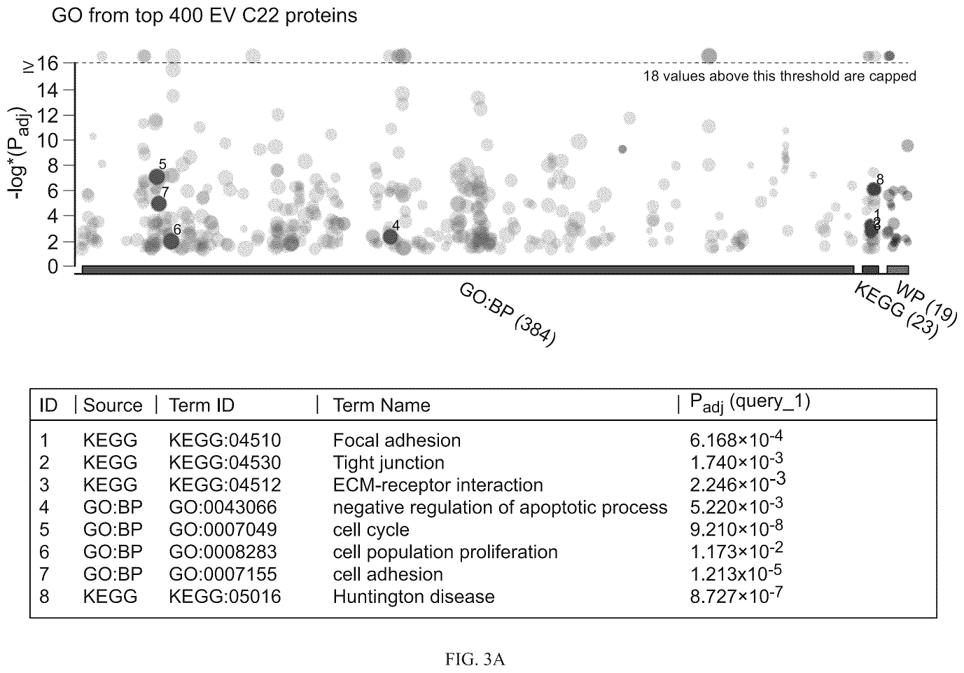

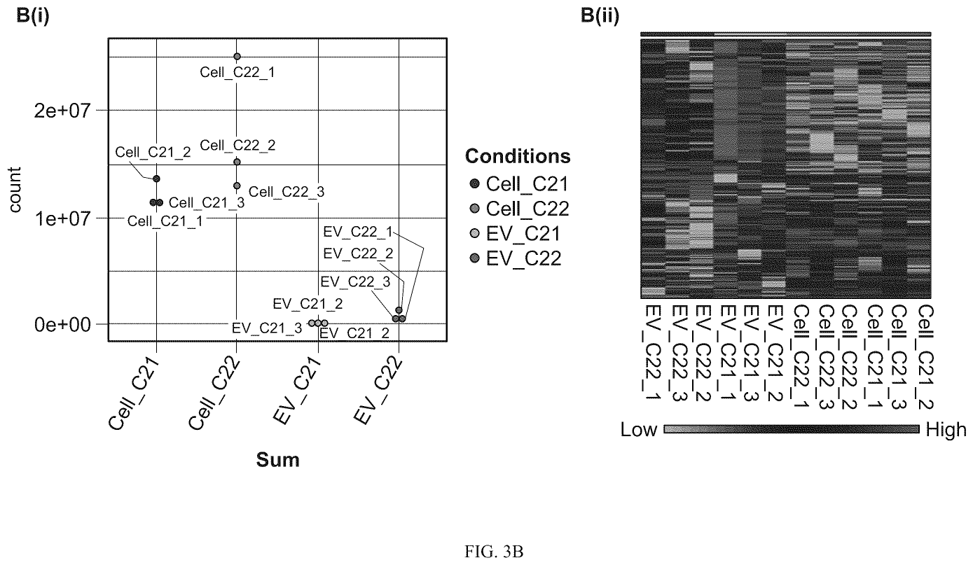

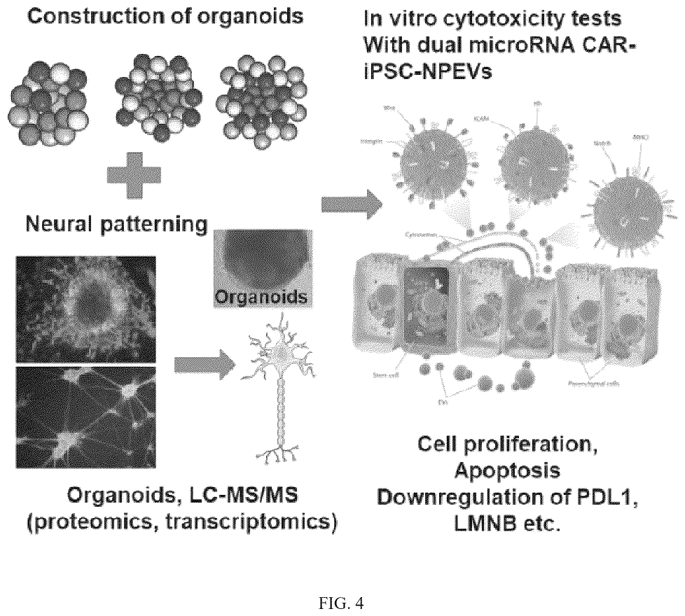

The isolation and characterization methods of EVs from human mesenchymal stem cells (MSCs) and iPSCs using Vertical Wheel bioreactors have been established. EVs from hiPSCs have been generated in Vertical Wheel bioreactors and performed proteomics analysis as well as miRNA-sequencing for protein and miRNA cargo characterization of hiPSC-EVs (FIG. 3A-3B). These results demonstrate the feasibility of implementing multi-omics tool to analyze protein and miRNA cargo of CAR-iPSC-NPEVs bio-manufactured from Vertical Wheel bioreactors.

### Multi-Omics Analysis of CAR-iPSC-Neutrophil Derived EVs at Day 12 and 21

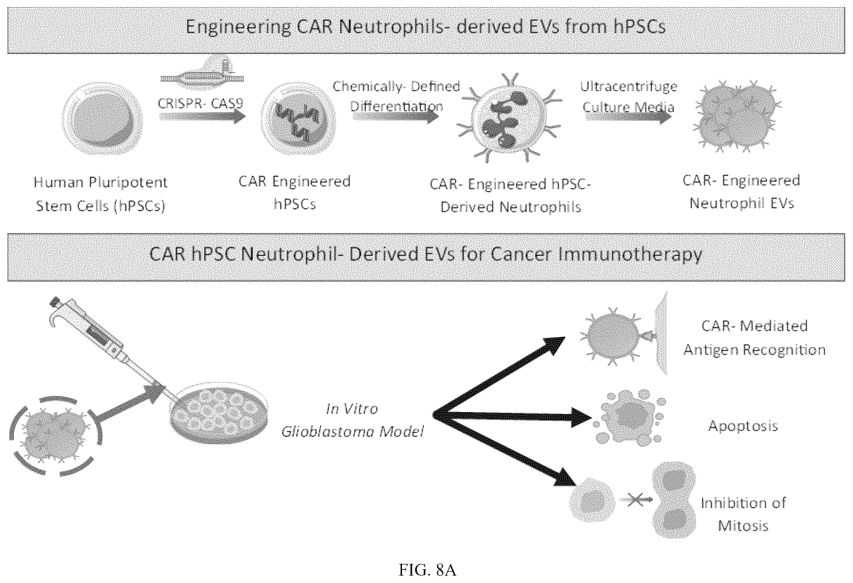

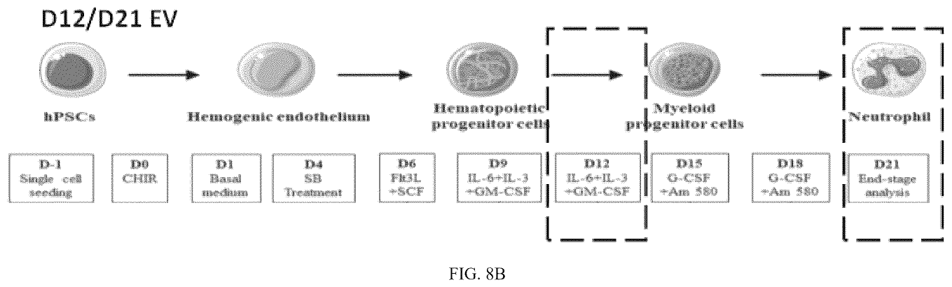

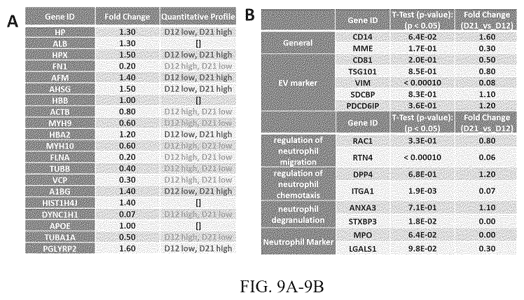

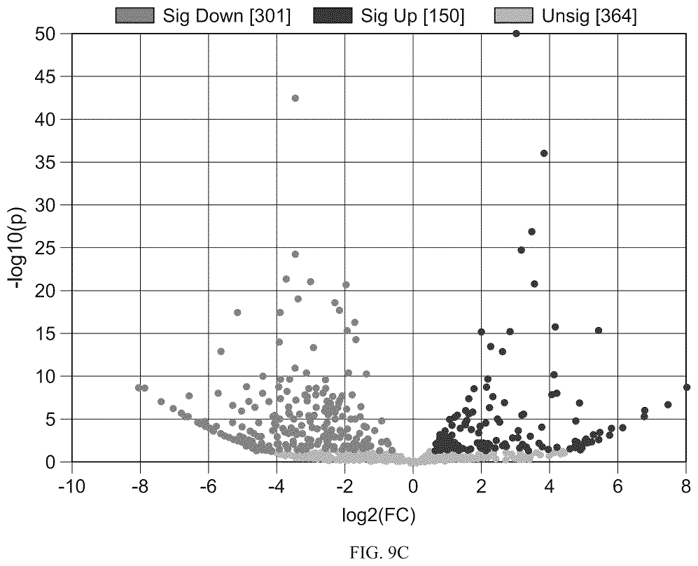

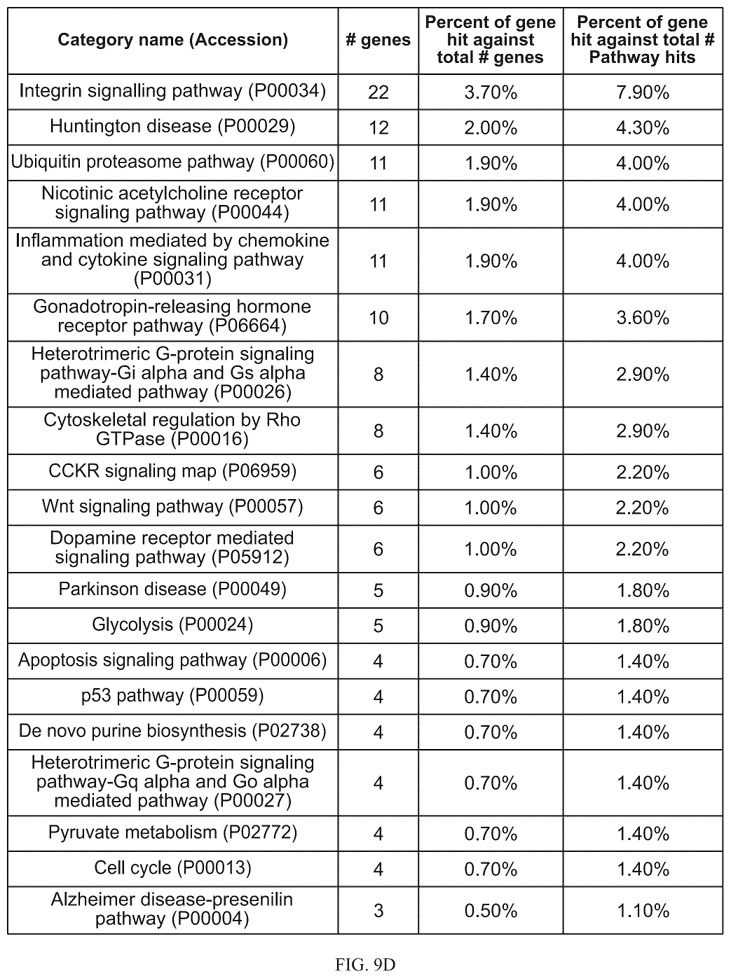

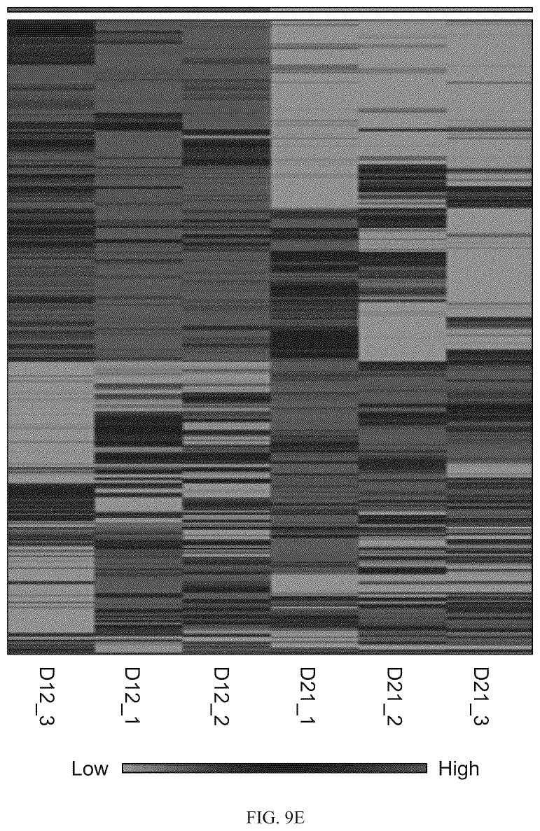

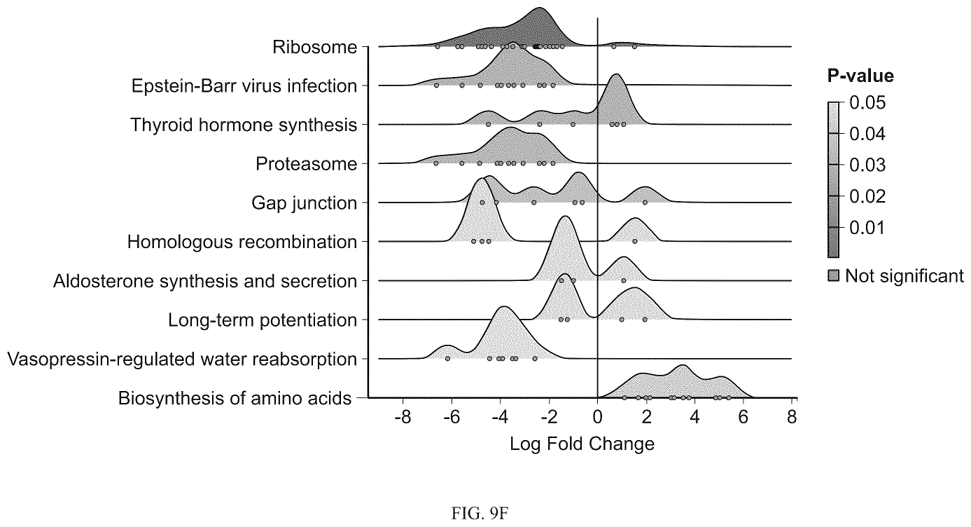

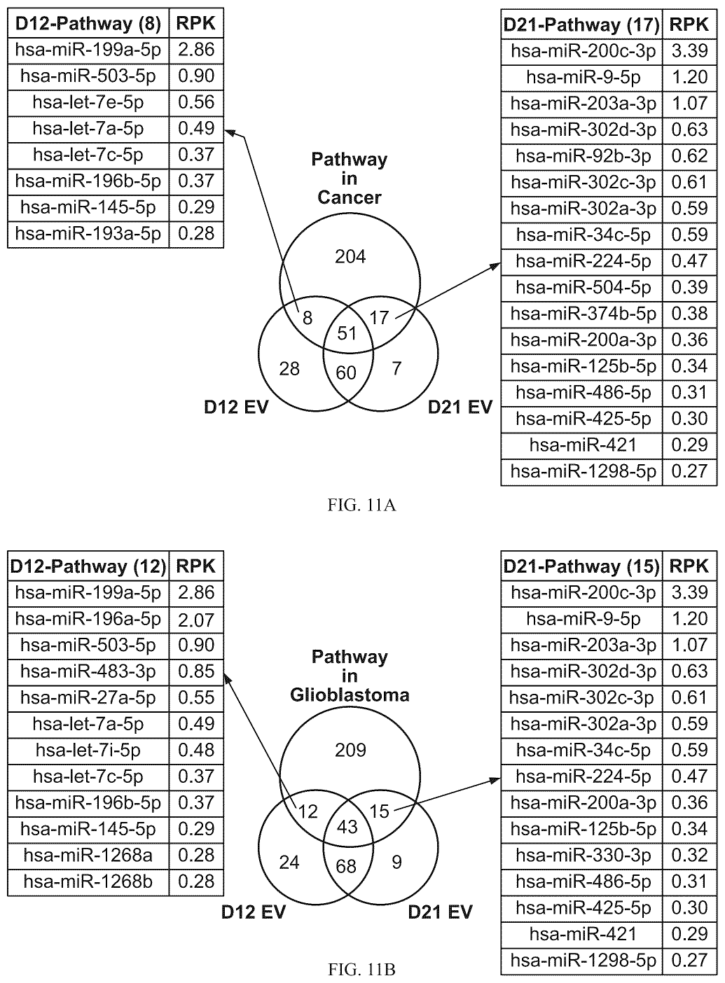

The protein cargo of day 12 and day 21 CAR-iPSC-Neu EVs was analyzed by proteomics and 456 differentially expressed proteins (DEPs) were identified (FIGS. 8B, 9A-9B). Compared to day 12, the neutrophil differentiation increased the protein content in the secreted EVs with HP (Haptoglobin), HPM, AHSG, HBA2, etc. The neutrophil markers and proteins related to regulation of neutrophil migration, chemotaxis, and degranulation were identified in EVs. The miRNA cargo of day 12 and day 21 EVs was analyzed by miRNA-sequencing and 74 differentially expressed genes (DEGs) were observed (FIG. 11B-11C). The top overlapped miRs with those related to pathway in GBM were identified. Kyoto Encyclopedia of Genes and Genomes (KEGG) pathway analysis for the top miRs (miR-21, 203a, 7, 145, 143, 125a, 22, 378a, 181a, 199a, 146a, 183, 92a, 34c, etc.) of day 21 EVs indicate that the miRNAs in the EVs are involved in mTOR signaling pathway, miRNAs in cancer, T cell receptor signaling pathway, cytokine-cytokine receptor interactions, Jak-STAT, Notch, Hedgehog signaling pathways, natural killer cell-mediated cytotoxicity, and various types of cancer-related pathways. These results demonstrate the feasibility to establish correlation between protein and miRNA cargo of CAR-iPSC-Neu EVs and the potential functions of EVs in GBM treatment.

### Biorepository of Fully Characterized Patient-Derived Glioma Stem Cell Enriched Lines, Patient-Derived Glioblastoma Organoids and GBM Murine Models:

A large biorepository of human-derived glioma stem cell enriched lines (>100) have been established, which have been leveraged to established murine GBM models. Further, GBM organoids have also been generated as previously published, recapitulating immune signatures of parental tumors.

### Experiments:

### LMNB2 and PD/L1 are Highly Overexpressed in Tumor Cells:

PD-L1 and Lamin B2 protein are oncogenic proteins that are overexpressed in tumor cells. PD-L1 and PD-1 have intracellular activity, and their overexpression predicts a poor prognosis. LMNB2 promotes tumor progression by increasing MCM7's chromatin binding and helicase activity. A resistant GBM model (U87MG cells, Sigma, 89081402) is tested. PD-L1 mRNA and LMNB2 mRNA and protein expression is examined. Primary GBM lines are also tested. High and low-expressing lines for PD-L1 and LMNB2 are used as glioma stem-cell enriched models.

### Bio-Manufacture EVs Using Vertical Wheel Bioreactors:

EVs are prepared by using PBS-MINI Mag Drive Bioreactor system (PBS Biotech Inc.) which has been fully established. Briefly CAR-IPSC-NP cells are grown in a-MEM media containing 20% EV-depleted fetal bovine serum (FBS) plus 5 ng/mL IL-15 and the media is collected at predetermined times and processed by the PEG method for extraction of EVs, which has been well established. The EVs are characterized by NTA analysis, TEM, Western blot, and AFM. The presence of CAR in the isolated EVs is confirmed by Western blot.

Loading miRNAs into CAR-IPSC-NPEVs:

Gene Pulser X Electroporator (Bio-Rad, USA) is used to electroporate miRNA into EVs. Briefly, EVs and miRNA are mixed in 400 μL of buffer at 4° C. After electroporation at 350 V and 150 μF, the mixture is incubated at 37° C. for 30 mins to fully recover the membrane of EVs. The four (4) miRNAs for PD-L1 are:
1. hsa-miR-3117;
2. hsa-miR-5193;
3. hsa-miR-4282; and
4. hsa-miR-548.

The LMNB2 miRNA (total of 4) used are:
1. hsa-miR-3148,
2. hsa-miR-4698;
3. hsa-miR-3133; and
4. hsa-miR-5700.

All these miRNA mimetics are synthesized by Integrated DNA technologies. These are also labeled with cy5.5 for fluorescent labeling for tracking and quantification. A total of 24 formulations (4 miRNAs for each PD-L1 and LMNB2) and 3 concentrations (25, 50, and 100 nM) are made.

Characterization of the assembled CAR-iPSC-NPEVs

### (1) Examine the Physical Properties:

Native polyacrylamide gel electrophoresis assays are used to characterize the encapsulation of miRNA, followed by imaging via a Typhoon FLA 7000. The atomic force microscopy and TEM are used to assess the structures of the EVs.

(2) Determine miRNA Loading Efficiency into EVs:

The concentration of miRNA EVs is measured by Cy5.5 fluorescent intensity.

### (3) In Vitro Pharmacokinetics for Cell Binding and Subcellular Trafficking Assays:

Confocal/fluorescent microscopy and flow cytometry are used to assess the binding and subcellular trafficking of EVs in glioblastoma cells.

(4) Examine Cell Survival and Proliferation Upon EVs Containing miRNA Treatment:

The GBM cells (in 2-D cultures) are incubated with various miRNAs and their EV formulations (at different doses of using 0.01, 0.1, and 1×10⁹ EVs/mL) for 48-72 hrs for 24-28 hrs. The cytotoxicity is examined by measuring changes in cell proliferation, apoptosis, cell morphology and colony potential, using methodologies from manufacturers or previous studies. Further, western blot and RT-PCR studies are conducted to assess the downregulation of PD-L1, LMNB2 and other apoptotic and proliferative markers (AMPK, integrin B3, SMAD5, Vimentin, P-28MAPK, BAX, BCL2 etc.).

(5) the Protein and miRNA Cargo of the CAR-iPSC-NPEVs

The protein and miRNA cargo of the CAR-iPSC-NPEVs are also studied by conducting proteomics and miRNA-sequencing using FSU Translational lab (TSL) core facilities.

### Proteomics Analysis of EV Cargo

Following the careful purification and analysis of EVs as described above, EV proteins are solubilized and digested with trypsin using the recently developed suspension trapping (Strap) sample preparation method for bottom-up proteomics. The Strap method combines the advantage of SDS-based protein extraction with rapid removal of detergents and other interfering agents (including PEG), trypsin digestion, and peptide clean-up prior to liquid chromatography with tandem mass spectrometry (LC-MS/MS). To maintain consistency and reproducibility, the Strap columns are obtained. Equal amounts of total protein from EVs or other vesicle populations harvested from the various cell lines are individually processed. The peptides generated following EV purification and Strap digestion are analyzed on a Thermo Q Exactive HF mass spectrometer with an online HPLC-nESI-LIT. Peptides are separated by two-dimensional (2-D) HPLC gradients prior to electrospray ionization and mass analysis and fragmentation. The resulting mass spectra are processed and peptides identified using the Mascot, Sequest, and MS Amanda search engines against a database containing the human Uniprot database with appended common repository of adventitious proteins. Statistical analyses is performed using the Scaffold proteomics software (Proteome Sciences) or the R statistical package.

mRNA and miRNA Profiling of CAR-EVs

In addition to proteomics, the RNA from EV samples are purified using the Qiagen miRNeasy and RNeasy kits and generate libraries using the NEBNext multiplex small RNA library kit following validation of the purity and quantity of RNA obtained. To ensure that the RNAs are of vesicle origin, EV samples are treated with RNAse prior to purification. The RNA samples are submitted for miRNA and mRNA deep sequencing. Statistically significant differences between sample groups are validated by RT-qPCR. All validated miRNA targets (e.g., miR-155, miR-21, etc.) are tested for their importance in AD-like phenotypes in the organoid models by delivering anti-miRNA oligonucleotides to cells or loading EVs with elevated levels of target miRNAs (e.g., miR-133) prior to incubation with organoid cultures.

### Selection Criteria and Outcomes:

From the above studies, the most effective formulation for each miRNA are selected based on the following criteria:
(a) isobolographic analysis showing combination index of <1;
(b) significant down regulation of PD-L1 and LMNB2 and other proteins (P<0.001);
(c) cytotoxicity at minimal dose of miRNA (<50 nM) in combination with miRNAs either alone or in the NPEVs formulation; and/or (d) in vitro pharmacokinetics.

In some embodiments, the engineered NPEVs comprise at least one miRNA selected from miR-5193 (PD-L1), miR-3133 (LMNB2), or a combination thereof. In some embodiments, the compositions described herein further comprise additional miRNAs. In some embodiments, the additional miRNAs comprise miR-34a, miR-424, or a combination thereof.

In some aspects, the formulation has three therapeutic components:
(a) CAR targeting GBM;
(b) the natural protein and miRNA cargo in CAR-NPEVs; and
(c) the loaded miRNAs.
   Evaluation of the Dual miRNA CAR-iPSC-NPEVs in 3D Human Brain Organoid Bearing GBM In Vitro.

CAR-iPSC-NPEVs loaded with PD-L1 and LMNB2 miRNAs reduce the cytotoxicity of 3D human brain organoid bearing GBM in vitro.

The brain tumor microenvironment (TME) consists of cancerous cells, stromal cells, and tissue-specific resident cells. Stromal cells include endothelial cells, fibroblasts, pericytes, and immune cells; whereas astrocytes, microglia, oligodendrocytes, and neurons make up the tissue-resident population of the brain. These cells are associated with the extracellular matrix (ECM), while physically shielded from the systemic circulation by the BBB. Human stem cell-derived brain organoids have recently been developed to provide the models for various cancers including GBM, which better recapitulate the TME than the 2D cultures. In vitro cytotoxicity assay of the formulated CAR-iPSC-NPEVs is performed using GBM patient-derived 3D cancer organoids generated as previously demonstrated.

### Studies:

Brain Organoids Derivation from Human iPSCs

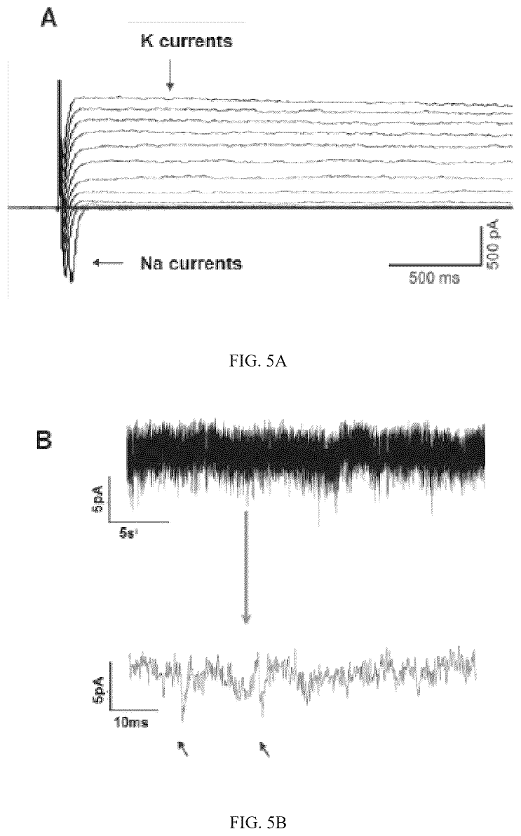

Recently, spheroids or organoids of various brain regions were generated from hiPSCs. By tuning sonic hedgehog signaling, spheroids of forebrain-cortical identity (expressing higher TBR1, a cortical layer VI marker) and hindbrain identity (expressing higher HOXB4 and Islet-1) were generated (FIG. 5A-5D). The cells also displayed Na+ and K+ currents and spontaneous excitatory post-synaptic currents (FIG. 5A-5D). These results demonstrate the feasibility of generating and characterizing brain-region-specific organoids. Microglia-like cells can be integrated in forebrain spheroids, and reduced TNF-α and TREM2 expression when treated with BAY11-7082 (a NF-KB inhibitor), in particular for the ventral groups. These results demonstrate the feasibility of integrating microglia with brain-region-specific organoids and use them for construction of human brain organoid bearing GBM.

### In Vitro Cytotoxicity of CAR-iPSC-Neu EVs on GBM Cells

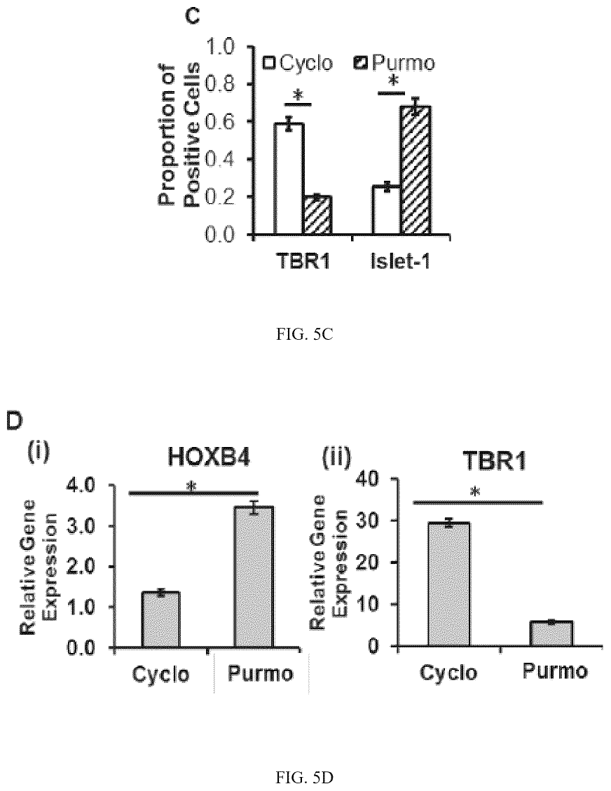

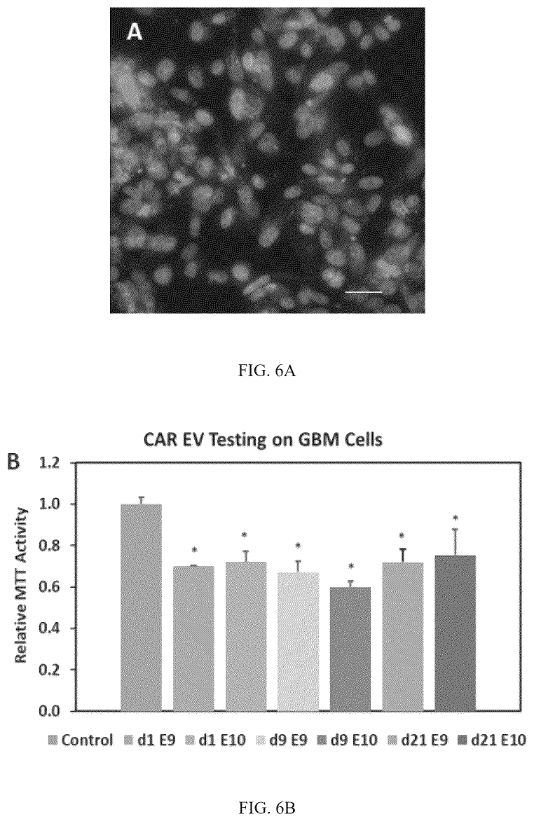

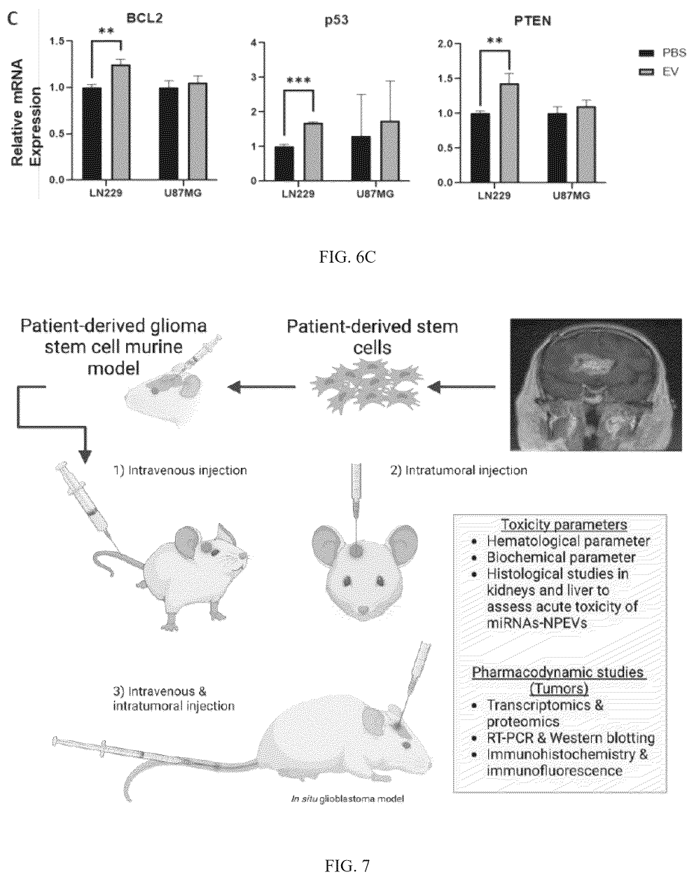

Cytotoxic effects of CAR-iPSC-Neu EVs were assessed using MTT assay wherein human GBM cells in ultra-low attachment 96-well plates were exposed to different doses of CAR-iPSC-Neu EVs (1×10⁹ and 1×10¹⁰ EVs/mL) for 48 h. CAR-iPSC-Neu EVs showed cytotoxic effects in the cells with reduced MTT activity of 60-75% (FIG. 6A-6C). The EV treatment also upregulated the genes that induce cell death. These results demonstrate the feasibility of cytotoxicity tests of dual microRNA CAR-iPSC-Neu EVs in 3D GBM organoid models.

### Experiments:

Construction of Human Brain Organoids Bearing GBM with Vascularization:

Human forebrain organoids can be formed by seeding healthy iPSCs and GBM patient-derived stem cell lines into ultra-low attachment 96-well plate (wp) (0.2-0.5×10⁵ cells per well) forming one organoid/well) and 24-wp (1-3×10⁵ cells per well) forming multiple organoids per well).

(1) For dorsal organoids, Cyclopamine (Cyclo) (a SHH inhibitor) and FGF-2 are used. The identity is defined by FOXG1, TBR1 (Cortical layer VI), CTIP2 (layer V), BRN2, and SATB2 (layer II-IV), for glutamatergic excitatory neurons, using immunostaining, flow cytometry, RT-PCR, and confocal microscope.

(2) For ventral organoids, SB+LDN+ purmorphamine (Purmo, a SHH activator), and IWP4 (a Wnt inhibitor) are used. The identity is defined by Nkx2.1, GABA, vGAT, and GABAergic inhibitory neurons. The astroglial spheres can be formed separately and co-cultured with the brain organoids (at a ratio of neuron:astrocyte=1:1) using previously established protocol. Vascular spheroids are generated from isogenic hiPSC lines using CHIR induction, which are fused with brain spheroids. Vertical Wheel bioreactors can also be used to generate brain organoids with promoted nutrient diffusion.

### Inclusion of Microglia (MG) Component in the Organoids:

The isogenic MG-like cells are differentiated from iPSCs as shown in previous publications. The cells are characterized by CD45, CD11b, and IBA-1 (day 20-23) for MG progenitors, and HLA-DR, CX3CR1, TREM2, TMEM119, and P2RY12 (day 30-50) for MG-like cells. MG-like cells are labeled with CellTracker Red. Then a fixed number of cells (0.2, 0.5, 1.0, 1.5, 2.0×10⁴) are added to low attachment 96-well plates containing one organoid per well for (1) dorsal organoids, (2) ventral organoids, and (3) fused dorsal-ventral organoids, at day 20-40. The microglia cellular distribution is examined with confocal microscopy. AMD3100, an antagonist of CXCR4, affects cell migration and interferes with CXCR4-mediated chemotaxis. AMD3100 is added at 100 nM to the dorsal or ventral organoids containing MG (pre-labeled). The cellular distribution of MG in hybrid organoids is examined using confocal microscopy. Immobilization of MG-like cells is observed after AMD3100 is added.

### Evaluating GBM Microenvironment in 3D Organoid Models:

The 3D cancer organoids are assessed using transcriptome analysis (to compare healthy and GBM organoids) and single-cell RNA-sequencing (for examining TME properties and heterogenous cell populations). The structure of the organoids are also examined by histology and confocal microscope (GBM organoids should have disorganized architecture). GBM-organoids should express higher SOX2, Ki67, CD99 in addition to GFAP and S100B. Transcriptional factors related to epithelial-mesenchymal transition (EMT) such as TGFB, STAT3, ZEB2, CXCR4 etc. are also be examined.

Cytotoxicity Tests of Dual miRNA CAR-iPSC-NPEVs in 3D GBM Organoid Models:

This part of experiments use primary tissue-derived and iPSC-derived cancer organoid models. The top 3 formulations of dual miRNA CAR-iPSC-NPEVs are tested. Briefly, the organoids are incubated with various miRNAs and their EV formulations (at different doses of using 0.01, 0.1, and 1×10⁹ EVs/mL) for 48-72 hrs for 24-28 hrs. The cytotoxicity is examined by measuring changes in cell proliferation, apoptosis, cell morphology and colony potential, using methodologies from manufacturers or previous studies. Further, western blot and RT-PCR studies are conducted to assess the downregulation of PD-L1, LMNB2 and other apoptotic and proliferative markers (AMPK, integrin B3, SMAD5, Vimentin, P-28MAPK, BAX, BCL2, etc.) after dual microRNA EV treatment.

### Outcomes:

The cancer organoids express the specific GBM markers. The gene profiles of the astrocytes and the MG-like cells exhibit the upregulation for the genes that are involved in anti-inflammation, tissue repair, and immunosuppressive factors. In particular, the dual miRNA CAR-iPSC-NPEVs work effectively against GBM cells in the 3D organoids. Viscoelastic hyaluronic hydrogels promote the brain tissue development.

Toxicological and Pharmacodynamic Evaluation of the Dual miRNA CAR-iPSC-NPEVs in GBM Models In Vivo.

CAR-iPSC-NPEVs loaded with PD-L1 and LMNB2 miRNAs penetrate BBB and reveal low toxicity in GBM models in vivo, and without wishing to be bound by any one theory, reduce the cytotoxicity of GBM cells in vivo.

To determine the function of CAR-iPSC-NPEVs, an in vivo xenograft mouse model via intracranial injection of luciferase-expressing patient-derived GBM cell lines are implemented to show antitumor cytotoxicity in vivo. Moreover, the toxicology and pharmacodynamics after the CAR-iPSC-NPEV injection are evaluated. For GBM models, it is also important to show the ability of the EVs to cross blood brain barrier (BBB) and to test uptake by the recipient cells. In addition to the immunotherapeutic benefits, the mechanisms for antitumor cytotoxicity of the CAR-iPSC-NPEVs are evaluated. While not wishing to be bound by any one theory, the mechanisms involved include phagocytosis, reducing reactive oxygen species (ROS), and neutrophil extracellular traps (NETs) formation. An experimental outline is shown in FIG. 7.

### Studies:

In Vitro BBB Model Derived from Human iPSCs

Results have shown the vascularization of forebrain organoids and the establishment of an in vitro BBB model. Transendothelial electrical resistance (TEER) analysis of the BBB model showed the value of 4158±305 (Ω·cm2). This model can be used for evaluating CAR-iPSC-NPEV transmigration.

### Patient-Derived GBM Murine Model

Leveraging the large biorepository of human and human derived specimens, which includes over 100 primary patient-derived glioma stem cell lines, GBM-murine models are established. These models can be used for evaluating anti-tumor ability of the engineered CAR Neu EVs.

### Experiments:

Tumor Uptake of miRNAs in EV Formulation:

To evaluate if NPEVs could reach tumors post i.v. administration, post intratumoral or combined i.v. + intratumoral, miR 3133-TYE-705 is synthesized (IDT laboratories) and incorporated in NPEVs using electroporation. NPEV-miR3133-TYE 705 is injected i.v., intratumorally, or both, into GBM-bearing mice or directly into GBM organoids formed in vitro.

Two hours after injection, tissue is isolated and sectioned (10 μm) with a cryotome. The sections are confocally imaged after DAPI is added (nuclear stain). No fluorescence is expected in control and miR3133-injected tumors. In some embodiments, NPEV-miR3133-TYE 705 is highly expressed in tumors. While not wishing to be bound by any one theory, high expression of NPEV-miR3133-TYE 705 in tumors indicates the targeting potential of NPEVs described herein.

### EV Phagocytosis, ROS, and NET Assays: Phagocytosis:

EVs (50 μg of total protein) are incubated with PKH26 solution (4 μL of PKH26 in 1 mL of diluent; Sigma-Aldrich, PKH26GL) at room temperature for 15 min; then, 1 mL of 5% bovine serum albumin (BSA) is added to stop the reaction, and the EVs are centrifuged at 120,000 g for 70 min and resuspended in DMEM. iPSC-derived forebrain organoids are incubated with the labeled EVs for 24 hours in 24-well plates, fixed for 15 min, blocked with 5% BSA, and then incubated overnight with anti-Nestin primary antibody (Abcam, ab197896) and for one hour with secondary antibodies. Nuclei are counterstained with DAPI (4′,6-diamidino-2-phenylindole; Vector, H-1200), and the cells are viewed with a confocal microscope. ROS production: The ROS levels of GBM cells with or without EV treatment can be measured using carboxy-H2DCFDA incubation with inducer tert-butyl hydroperoxide (ROS detection kit). NET formation: This is measured using propofol treatment followed by PicoGreen staining.

### BBB Transmigration Assay:

In vitro BBB model using transwell insert is constructed as shown in preliminary results from iPSCs, in DMEM-F12 medium containing 10% EV depleted FBS. 0.1-1×10° NPEVs labeled with PKH26 are added to the upper chamber. For cytotoxicity analysis, the 2×10⁴ GBM cells are seeded at the lower chamber 12 hours before adding EVs. The tumor cell viability is determined by flow cytometry. The labeled EVs are examined in the U87MG cells by fluorescence microscopy.

### Toxicological Evaluation in Mice:

One of the most important criteria for using EV miRNA as a nano delivery platform is its safety profile. To this end, toxicological assessments of EVs are performed in mice. For cytokine induction study, BALB/c mice are injected via tail vein with phosphate buffered saline (PBS) or EV/PD-L1 or LMNB2 at 20 μg/kg. The plasma is prepared 3 hrs post-injection and analyzed for mouse TNF-α and IL6 using an enzyme-linked immunosorbent assay. For a 7-day repeat dose study, BALB/c mice are injected with PBS or EVs with PD-L1 or LMNB2 at 20 μg/kg. The mice are treated once every 48 hrs for 7 days (four doses total). The mice are monitored for clinical signs. On day 7, three hours after the fourth injection, the mice are euthanized, and blood samples are collected by cardiac puncture for standard panel clinical chemistry and clinical pathology analysis. The gross pathology and organ weights are recorded (including spleen, lymph nodes, liver, kidneys). 24 mice are used for toxicological evaluation (10 weeks old; n=6 mice/group; both sexes; 2 types of tests).

PD-L1 and LMNB2 miRNA Encapsulated EVs for Cancer Marker Expression.

Briefly, 5 million cells or 1×10⁸ NPEVs/kg or a similar dose of human dermal fibroblasts (hFB)-EVs are transplanted in NOD SCID gamma mouse (NSG) immunodeficient mice (Jackson Labs) and when tumors are 800 mm3, treatment is started with miR-5193 (knockdown PD-L1) and miR-3133 (knockdown LMNB2) either alone (Group I and II) or in NPEVs (Group III and IV), along with vehicle control (group V) (n=6 per group; 3 administration routes; total=90 mice). For preparing NPEV formulations, NPEVs (100 μL, approximately 1× 10⁸ per kg) and miRNA (5 μM) are mixed at 4° C. After electroporation at 350 V and 150 μF, the mixture is incubated at 37° C. for 30 mins to fully recover the membrane of EVs. Further, EVs are injected to mice for one week every alternate day (3 doses) by i.v, intratumoral or combined routes. The tumors are then collected, and downregulation of various proteins are assessed by western blotting and RT-PCR. In some embodiments, miR-3133 has no effect on the downregulation of LMNB2. In some embodiments, an NPEV formulation comprising miR-3133 (3133-EXO) significantly downregulates LMNB2 protein expression. In some embodiments, miR-5193 an NPEV formulation comprising miR-5193 (5193 EXO) downregulates PD-L1, LMNB2, or a combination thereof. Without wishing to be bound to any one theory, dual down-regulation of PD-L1 and LMNB2 suggest the role of PD-L1 downregulation is connected to other proteins. Moreover, the roles of these miRNAs in regulating AMPK (upregulated), Integrin 3, SMAD 5, Vimentin, p-38 MAPK, and BCL2, SOD2 (reduced) which are involved in cancer development, metastasis, and chemotherapeutic resistance, are also evaluated.

Pharmacodynamic Studies with GBM Animal Model:

Further, the best formulation is studied in xenograft GBM model based on NSG mice. In some embodiments, the dual miRNA formulation is the most effective, and groups are:
(a) CAR-NPEVs;
(b) CAR-NPEVs containing dual miRNAs; and
(c). scramble miRNA-containing NPEVs (n=8, 3 administration routes, total 72 mice).

The tumors are excised at the end of the study and analyzed using western blot, proteomics, mRNA and miRNA-sequencing, RT-PCR, and histological techniques. Crosstalk between PD-L1 and LMNB2 in treating GBM is evaluated. To better study the interactions between CAR-Neu EV/siRNAs and immune cells, syngeneic mice are used and the immune landscape is profiled in glioma TME with or without CAR-Neu EV/siRNAs treatment. Their therapeutic efficacy will also be determined in syngeneic mice.

### Statistical Analysis:

Each treatment group includes 10 animals. This sample size was determined based on a power analysis, based on the most effective treatment having a 40% reduction in tumor growth rate when compared to the control, with a power of 85%. Assuming the standard deviation of the outcome measurements is equal to the difference between means in the two samples, a two-sided, two-sample, t-test with equal variance was conducted. There were no sex-based differences in the expected mechanism of action. Post-hoc analyses are conducted to see if there are any gender differences, and subsequent samples are adjusted accordingly.

### Example 2. Genetically Engineered Extracellular Vesicles with Chimeric Antigen Receptor of Human Stem Cell-Derived Neutrophils for Treating Glioblastoma

### Introduction

Glioblastoma Multiforme (GBM) is the most prevalent and malignant subtype of glioma, accounting for 80% of brain tumors and having a median survival of 15 months post-diagnosis. Current treatments for GBM include surgical resection, radiation therapy, and chemotherapy with temozolomide. This treatment approach only extends median survival by a few months and is also subject to side effects. Glioblastoma presents unique challenges in cancer treatment due to its location. In addition to the difficulties innate in treating cancer, glioblastoma treatments can be systematically excluded from the tumor by the blood-brain barrier, which highly regulates material transport into neural tissue. This creates a need for novel treatment methods that are capable of transport into neural tissue while also localizing to the tumor microenvironment and inducing tumor lysis. Research into such methods has been abundant, and have produced therapeutic alternatives including vaccinations, virotherapy, and immune cell therapy.

Immune cell therapy is a recently developed cancer therapy that uses a patient's immune cells to eliminate cancer. This involves the employ of chimeric antigen receptors (CARs), or genetically engineered proteins with a tumor-antigen recognition domain and an intracellular immune activating domain. T-cells are the most used cell type in immune cell therapy, though NK cells and macrophages have also been used in investigative trials. Recently, a turn has been made from engineering patient-derived immune cells to induced pluripotent stem cells (iPSCs) for immune cell therapy. iPSCs are stem cells capable of differentiating into any cell type within the body, including immune cells, offering a greater and more uniform cell yield than patient-derived cells. In addition, the ability of iPSCs to differentiate into any cell type in the body allows for immune cells that have short half-lives ex vivo or that are resistant to gene editing to be employed in CAR immune therapy. This includes novel cell types such as neutrophils and more controlled cell subtypes such as γδ T-cells and mucosal associated invariant T-cells.

Though immune therapy is shown to be effective for treating cancer, it is not without drawbacks. These include Cytokine Release Syndrome, ICANS, and on-target/off-tumor toxicity (OTOT), which can, in extreme cases, lead to patient death. Efforts have been taken to reduce the severity of off target effects in immune therapy via cotreatment with anti-inflammatory drugs and engineering strategies to limit CAR activity, with limited success. Another strategy to limit off-target effects in CAR immune therapy turns from the use of CAR engineered cells to CAR cell-derived extracellular vesicles. Extracellular vesicles (EVs) are small, membrane bound secretions that have paracrine function via the proteins and miRNAs they carry from their parent cells. Extracellular vesicles come in three classes based on their size and origin: apoptotic bodies, microvesicles, and exosomes. Exosomes, the smallest subclass of extracellular vesicles, retain the functional properties of their parent cells while also having function parent cells may not have, such as barrier permeability and avoiding allogeneic immune responses.

Biomedical research in cancer treatment has shown that EVs derived from immune cells carry inflammatory and pro-apoptotic cargos native to their parent cells. In vivo cancer modelling shows that these EVs localize to the tumor microenvironment and mediate tumor lysis. In addition, immune cell derived EVs can bypass the blood-brain barrier and inducing tumor lysis. Furthermore, studies done on EVs derived from CAR T-cells show that EVs present CARs on their surface just as their parents do. CAR presentation introduced targeting function into EVs, increasing their localization to tumor cells displaying the CAR's targeted antigen, enhancing tumor cytotoxicity and decreasing off-target uptake. These effects are also observed in CAR NK-cell derived EVs, suggesting these effects are not limited to a specific cell type. Recently, neutrophils presenting chlorotoxin, an anti-MMP2 receptor, have been engineered as a potential glioblastoma therapy. However, the cytotoxic effects of EVs from these cells have not been examined in a glioblastoma model. This study seeks to fill this gap in knowledge using in vitro GBM models to assess CAR neutrophil-derived EV cytotoxicity and blood-brain barrier permeability, while using proteomic and genomic analysis to examine changes in EV cargo that affect their cytotoxicity.

### Materials and Methods

The DNA for the CAR-CLTX was inserted into the AAV1 safe locus of induced pluripotent stem cells, which were differentiated into neutrophils. Conditioned media were collected along the differentiation and went through the extraPEG EV isolation. EVs were quantified by nanoparticle tracking analysis, electron microscopy, and western blot. Proteomics analysis of protein cargo and microRNA (miRNA) sequencing of the EVs were performed for day 12 and 21 samples. The cytotoxicity of the EVs was performed with U87MG and LN229 glioblastoma cells in 2D culture and 3D organoids.

CAR-hiPSC Differentiation into Neutrophils

CAR engineering and neutrophil differentiation was performed as shown in a previous study.

### EV Isolation

To isolate EVs, a differential centrifugation using polyethylene glycol (PEG) precipitation was employed as seen in our previous publications. Conditioned medium was centrifuged at 500 g for 5 min (Eppendorf, Centrifuge 5810 R, Germany). The supernatant was centrifuged at 2000 g for 10 min and the pellet discarded. The supernatant was then centrifuged at 10000 g for 30 min at 4° C. The pellet was discarded and a PEG solution (24% w/v in 1.5 M NaCl) was added to the supernatant in a 2:1 ratio to give a solution of 8% PEG and incubated at 4° C. overnight. After incubation, the solutions were centrifuged at 3000 g for 1 h. The supernatant was discarded, and the pellet was allowed to dry for 15 min. The pellet was then resuspended in particle-free milliQ PBS and ultracentrifuged at 127,000 g for 70 min at 4° C. (Beckman Coulter, Optima MAX-XP Ultracentrifuge, CA). The supernatant was discarded, and the pure EV pellet was suspended in 100 μL PBS and shaken (Eppendorf, ThermoMixer C, Germany) for 15 min at 1500 rpm.

### Nanoparticle Tracking Analysis (NTA)

NTA was performed on isolated EVs to characterize EV size distribution and concentration using a Nanosight LM10-HS instrument (Malvern Instruments, Malvern, UK), which is configured using a 488 nm blue light and sCMOS camera. EVs were diluted 1:1000 in particle-free PBS before NTA. Three videos of 60 s duration were taken, with the camera level set to 11 and the detection threshold set to 5. Between each reading, the laser compartment was washed with 10% ethanol, followed by a wash with particle-free water. The videos were analyzed by NTA 3.4 software to calculate mean and mode particle size, as well as particle concentration.

### Transmission Electron Microscopy (TEM)

Electron microscopy imaging was used to confirm the morphology and size of EVs as shown in our previous studies. Briefly, EV isolates were resuspended in 30 μL of filtered PBS. For each sample preparation, intact EVs (15 μL) were dropped onto Parafilm. A carbon coated 400 Hex Mesh Copper grid (Electron Microscopy Sciences, EMS) was positioned using forceps with coating side down on top of each drop for 1 h. Grids were rinsed three times with 30 μL filtered PBS before fixed in 2% PFA for 10 minutes (EMS, EM Grade). The grids were then transferred on top of a 20 μL drop of 2.5% glutaraldehyde (EMS, EM Grade) and incubated for 10 min. Samples were stained for 10 min with 2% uranyl acetate (EMS grade). Then the samples were embedded for 10 min with a mixture of 0.13% methyl cellulose and 0.4% uranyl acetate. The coated side of the grids were left to dry before imaging on the Transmission Electron Microscope HT7800 (Hitachi, Janan).

### Western Blot for Exosomal Markers

EV and cell samples were lysed in radio-immunoprecipitation assay (RIPA) buffer (150-mM sodium chloride, 1.0% Triton X-100, 0.5% sodium deoxycholate, 0.1% sodium dodecyl sulphate, 50-mM Tris, pH8 and 1× Thermo Scientific™ Halt™ Protease Inhibitor Cocktail) (Thermo Fisher Scientific Inc., Waltham, MA, USA). Samples were incubated for 20 min on ice, and spun down at 14,000 rpm for 20 min. The cleared supernatants were collected, and a Bradford assay was carried out to determine protein concentration. Protein lysate concentration was normalized, and denatured at 100° C. in 2×Laemmli Sample buffer for 5 min. About 3-10 μg of proteins were loaded into each well. Proteins were separated by 12% Bis-Tris-SDS gels and transferred onto a nitrocellulose membrane (Bio-Rad, Hercules, CA. USA). For the detection of non-phosphorylated proteins, the membranes were blocked for 1 h in 1% w/v non-fat dry milk in Tris-buffered saline (10-mM Tris-HCl, pH 7.5 and 150-mM NaCl) with 0.1% Tween 20 (v/v) (TBST). Membranes were incubated overnight in the presence of the primary antibodies diluted in blocking buffer at 4° C. Afterwards, the membranes were washed four times with TBST and then incubated with a horseradish peroxidase (HRP)-conjugated secondary antibody at 1:5000 for 1 h. The blots were washed and imaged on the Biorad ChemiDoc Imaging System.

Liquid Chromatography with Tandem Mass Spectrometry (LC-MS/MS) Based Proteomics Analysis of EV Protein Cargo

The EVs (day 12 and day 21) were isolated using ExtraPEG and then extracted for proteins. Based on protein quantification results, up to 24 μg proteins were isolated on S-trap micro column (Protifi, K02-micro). The isolated proteins were alkylated and digested on column based on manufacturer's instructions. Then all the samples (triplicate for each group) were vacuumed dried and submitted to FSU Translational Science Laboratory. The samples were analyzed on the Thermo Q Exactive HF as previously described. Briefly, resulting raw files were searched with Proteome Discoverer 2.4 using SequestHT, Mascot and Amanda as search engines. Scaffold (version 5.0) was used to validate the protein and peptide identity. Peptide identity was accepted if Scaffold Local false discovery rate (FDR) algorithm demonstrated a probability greater that 99.0%. Likewise, protein identity was accepted if the probability level was greater than 99.0% and contained a minimum of two recognized peptides. Gene Ontology (GO) annotation was carried out by g: Profiler.

Small RNA Sequencing for EV miRNA Cargo Analysis

EV-associated miRs were isolated and sequenced in triplicate. EV samples were treated with RNase (ThermoFisher, AM2294) to final concentration of 50 ng/mL, at room temperature for 30 mins. RNase inhibitor (NEB, M0314) and PCR grade water were added to EV samples to make a total volume of 200 μL. miRs were isolated by adding 600 μL Trizol LS (ThermoFisher, 10296010) according to manufacturer's instruction. To increase the yield of small RNAs, three volumes of 100% ethanol and linear acrylamide (VWR, 97063-560) were used instead of isopropyl alcohol and incubation time was also increased to overnight at −20° C. The isolated RNAs were quantified by Qubit microRNA assay kit (ThermoFisher, Q32880). Small RNA libraries were generated with NEBNext Multiplex Small RNA Library Prep Set for Illumina (NEB; E7300). To increase yield and prevent primer/adaptor dimer, 3′ SR primer was diluted to 1:5 and increase ligation time to overnight at 16° C. Similar to mRNA-seq library preparation, HS DNA chip and KAPA library quantification kit were used before submitting to sequencing by Illumina NovaSeq 6000 in Florida State University College of Medicine Translational lab.

### RNA-Seq Data Analysis

Raw data for miR-seq were submitted to OASIS online miR analysis tool to identify small RNAs on Human reference genome hg38. Differential expressed miRs were analyzed by both OASIS and miRNet using default settings. RNA-seq data was analyzed by NetworkAnalyst 3.0. Genes with counts less than 10, variance less than 10% and unannotated were filtered and normalized by Log 2-counts per million. Differentially expressed genes (DEGs) were identified by DEseq2. Heatmap of globe differential expressed genes and gene enriched pathways were also visualized by the same online tool.

### GBM Cell Culture

Two glioblastoma cell lines were used in testing: U87MG and LN229. U87MG cells (Millipore Sigma, 89081401-1VL) were seeded into T-flasks at a seeding density of 30,000 cells/cm² and cultured with EMEM (EBSS)+2 mM Glutamine+1% Non-Essential Amino Acids (NEAA)+1 mM Sodium Pyruvate (NaP)+10% Fetal Bovine Serum (FBS) (Atlanta Biologicals, Lawrenceville, GA) and 1% Penicillin/Streptomycin (Life Technologies, Carlsbad, CA) in a standard incubator at 37° C. with 5% CO2 and 20% 02. At 90% confluence, cells were detached from culture plate using.25% trypsin at 37° C. for 5-10 min. Harvested cells were replated at 30,000 cells/cm². LN229 cells (ATCC, CRL-2611) were seeded into T-flasks at a seeding density of 30,000 cells/cm² and cultured with DMEM+5% FBS+1% Penicillin/Streptomycin in a standard incubator at 37° C. with 5% CO2 and 20% 02. At 90% confluence, cells were detached from culture plate using.25% trypsin at 37° C. for 5-10 min. Harvested cells were replated at 30,000 cells/cm².

### CAR-EV Uptake Analysis by GBM Cells

To evaluate EV uptake, EVs were labelled with PKH26 (Sigma-Aldrich, PKH26GL). Briefly, a PKH26 labelling solution was made using 4 μL of PKH26 in 1 mL of dilutant. EVs were labelled in the PKH26 solution for 15 min at room temperature. The reaction was then quenched with 1 mL of 5% BSA solution. EVs were then centrifuged at 127,000 g for 70 min and resuspended in PBS. For uptake evaluation by ICC, glioblastoma cells (either U87MG or LN229) were seeded into 96 well plates at a density of 1,000 cells/well and allowed to attach overnight. PKH26 labelled EVs were added to cell culture medium and allowed to sit for 24 h. Afterwards, growth medium was removed, cells were washed with PBS and fixed with 4% paraformaldehyde for 15 min at room temperature. Cell nuclei were stained with Hoescht-33342 and then imaged.

For analysis by flow cytometry, cells were seeded into 12 well plates at a density of 100,000/well. Labelled EVs were added to cells and allowed to incubate for 24 h. Afterwards, cells were detached using 0.25% trypsin, centrifuged at 500 g, resuspended in PBS, and analyzed on a FACS Canto flow cytometer. Data analysis was performed using Flowjo software.

### In Vitro Cytotoxicity Analysis

Cytotoxicity testing began with 1,000 cells/well seeded into a 96 well plate, either TCP treated (2D assays) or ultra-low attachment (3D assays) and allowed to adhere or aggregate overnight. EVs or PBS were added to cell cultures 1 day after passage. EVs were added to the culture on day 3 and 5 after passage. Assays were performed on day 7.

### 3-(4,5-Dimethylthiazol-2-yl)-2,5-diphenyltetrazolium bromide (MTT) assay

MTT stock solution (Sigma, 5 mg/mL) was diluted in cell culture media at 1:10 and was added to 96 well plates containing tumor cells and allowed to react for 2-4 hours. After sufficient incubation time, formazan crystals were gently washed with PBS and solubilized in DMSO. Samples were transferred into microcentrifuge tubes and centrifuged at 800 g for 5 minutes. 50 μL of sample supernatant was transferred to a well of a 96 well plate and read on a microplate reader (BioRad Laboratories, Hercules, CA, USA) at 500 nm.

### Lactate Dehydrogenase (LDH) Release Assay

To measure LDH release, the CyQUANT LDH cytotoxicity kit (Thermo Fisher) was used. According to the manufacturer's protocol, 50 μL of culture medium was removed from cell culture and placed into a well of a 96 well plate. 50 μL of reaction mixture was added to each media sample and allowed to incubate for 30 minutes at 37° C. protected from light. After 30 minutes, 50 μL of stop reaction solution was added to each well. The absorbances at 490 nm and 680 nm were measured by the microplate reader (BioRad Laboratories, Hercules, CA, USA). The 680-nm absorbance value was subtracted from the 490-nm absorbance. The cytotoxicity was calculated using the formula: % cytotoxicity= [(EV-treated LDH activity-spontaneous LDH activity)/(maximum LDH activity-spontaneous LDH activity)]*100%.

### EV Uptake in 3D Tumor Spheroids

3D aggregated cells were cocultured with PKH26 labelled EVs on day 1 of aggregate culture. EV treatment continued over the course of 1 week with additions of half a volume of media and half the initial EV dose every 3 days. Live aggregates were imaged on a epifluorescence microscope with Zeiss Axio Observer 7.

### Cytotoxicity Assay in 3D Tumor Spheroids

3D aggregated cells were treated with EVs on day 1 of aggregate culture. EVs were added every 3 days at half the initial dose in combination with half a volume of initial media. After 2 weeks, aggregates were dissociated with 0.25% trypsin/EDTA (Invitrogen) and stained with Trypan Blue (Sigma) before counting all cells in the viewing field.

### Reverse Transcription-Polymerase Chain Reaction (RT-qPCR)

Cells for both glioblastoma cell lines were seeded into 12 well plates at 2 million cells per well and allowed to adhere overnight. EVs were added to the EV+ groups at 4.5 E11 EVs per well and allowed to sit for 4 days. On day 4, cells were dissociated via trypsin, washed with PBS, and their mRNA isolated using the RNeasy Plus kit (Qiagen, Hilden, Germany) according to vendor instructions. Isolated RNA was cleaned using the RNA Clean and Concentrator kit (Zymo Research, California, USA) according to manufacturer's instructions. Reverse transcription was carried out using 2 μg of total RNA, anchored oligo-dT primers (Operon) and Superscript III (Invitrogen). Primers were designed using the software Oligo Explorer 1.2 (Genelink). RT-PCR reactions were performed on an ABI7500 instrument (Applied Biosystems), using a SYBR Green PCR Master Mix. Fold variations in gene expressions were quantified using the delta-delta Ct approach:

$${2}^{- ( Δ {C}_{t treatment} - Δ {C}_{t Control} )} ,$$

which is based on the comparison of the target gene (normalized to GAPDH) among different conditions.

### Statistical Analysis

All experiments were performed in triplicate (n=3), and representative data were reported. Experimental results were expressed as means±standard deviation (SD). Statistical comparisons were performed by one-way ANOVA and Tukey's post hoc test for multiple comparisons, and significance was accepted at p<0.05. For comparisons of two conditions, student's t-test was performed for the statistical analysis.

### Results

### EV Isolation and Characterization

FIG. 8A-8B shows the protocol of CAR engineered iPSC differentiation into neutrophils.

Exosomes from various times throughout the differentiation protocol of the CLTX-CAR displaying iPSCs were isolated according to the ExtraPEG Method. The size distributions and zeta potential of isolated samples were calculated by NTA (FIG. 2A (i-ii)). Exosomes were all found to have a mean diameter under 200 nm and zeta potentials ranging between −10-−30 mV (FIG. 2A (iii-iv)). No significant differences between sizes were observed between samples. Transmission electron microscopy of isolated exosomes (day 21) revealed cup shape morphology indicative of exosome identity (FIG. 2B). Western blot analysis of exosome sample revealed that all samples were positive for exosomal markers and negative for the Golgi marker calnexin (FIG. 2C).

The release of exosomes per mL of media was also evaluated using NTA. This revealed that throughout the differentiation protocol, exosomes are secreted in high numbers at early stages, decreasing as cells begin differentiation until they near terminal differentiation (FIG. 2D).

### Proteomic Analysis of CLTX-CAR-Exosomes

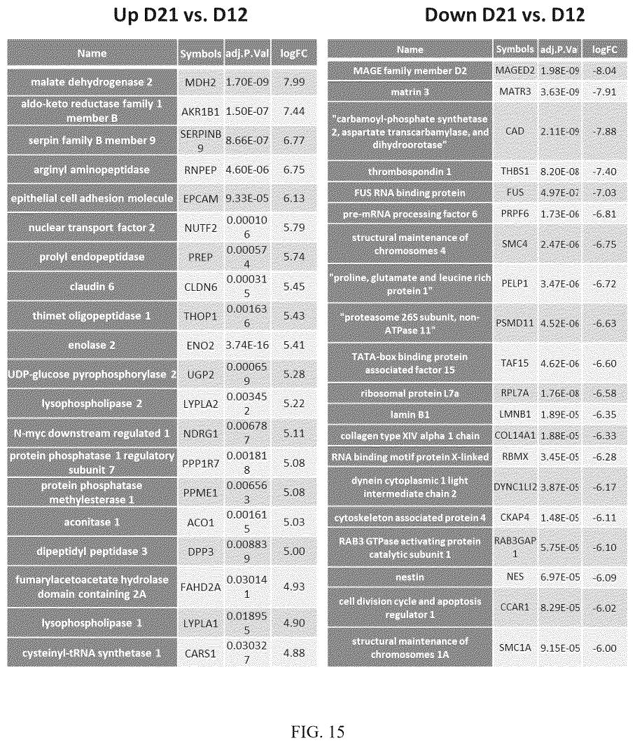

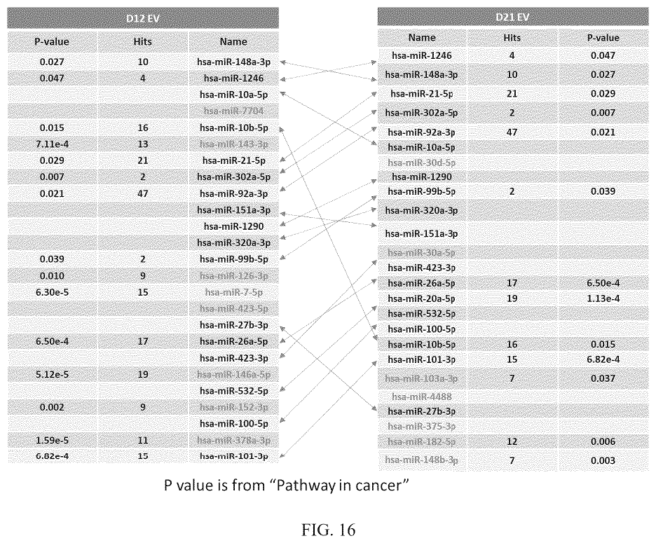

Exosome samples from day 12 and day 21 of the differentiation protocol were subject to proteomic analysis to track differences in protein cargo as cells develop into neutrophils (FIG. 9A). The differential expression of proteins involved in neutrophil maturation and EV biogenesis were analyzed. In both cases, protein expression was upregulated in d21 EVs compared to d12 EVs, suggesting that EVs mirror the maturity of their parent cell and that EV biogenesis through the ESCRT pathway is increased in mature neutrophils (FIG. 9B). Volcano Plotting shows that 301 proteins are significantly downregulated in d21 EVs vs. d12 EVs, 155 proteins are significantly upregulated in d21 EVs vs. d12 EVs, and 561 proteins experienced no significant change between the samples (FIG. 9C). The top 10 DEPs upregulated in d21 EVs are MDH2, AKRIB1, SERPINB9, RNPEP, EPCAM, NUTF2, PREP, CLDN6, THOP1, and ENO2. The top 10 DEPs downregulated in d21 EVs vs. d12 EVs are MAGED2, MATR3, CAD, THBS1, FUS, PRPF6, SMC4, PELP1, PSMD11, and TAF15 (FIG. 15). Heatmap analysis reveals that d12 and d21 EV groups are distinct populations with great reliability (FIG. 9E).

Kyoto Encyclopedia of Genes and Genomes (KEGG) analysis identified signaling pathways enriched in d21 EVs vs. d12 EVs. The top pathways enriched in d21 EVs include the integrin signaling pathway, Huntington disease, ubiquitin proteasome pathway, nicotinic acetylcholine receptor signaling pathway, cytokine signaling pathway, and gonadotropin-releasing hormone receptor pathway. Apoptosis signaling and p53 pathways were also differentially regulated between d12 and d21 EVs (FIG. 9D). Ridgeline plotting of proteomic analysis reveals pathways that are significantly downregulated in d21 EVs vs. d12 EVs include ribosome, Epstein-Barr virus infection, proteasome, gap junction, and vasopressin-regulated water reabsorption (FIG. 9F). Overall, these results suggest that the proteome of d21 EVs will modulate cancer apoptosis and proliferation.

CLTX-CAR-Exosome miRNA Cargo by Small RNA-Seq

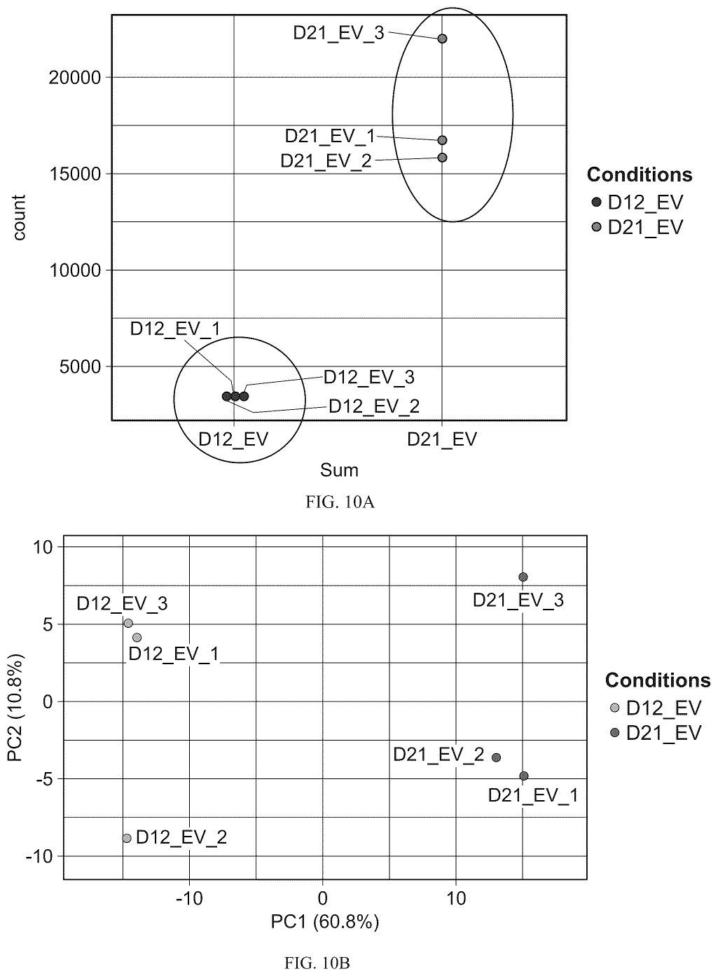

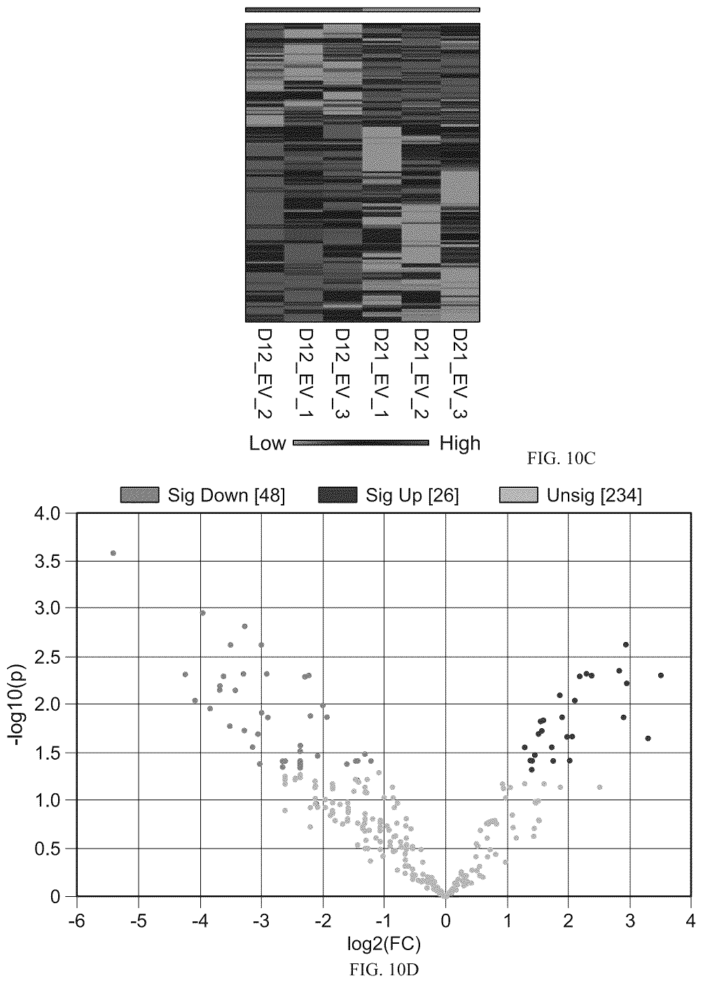

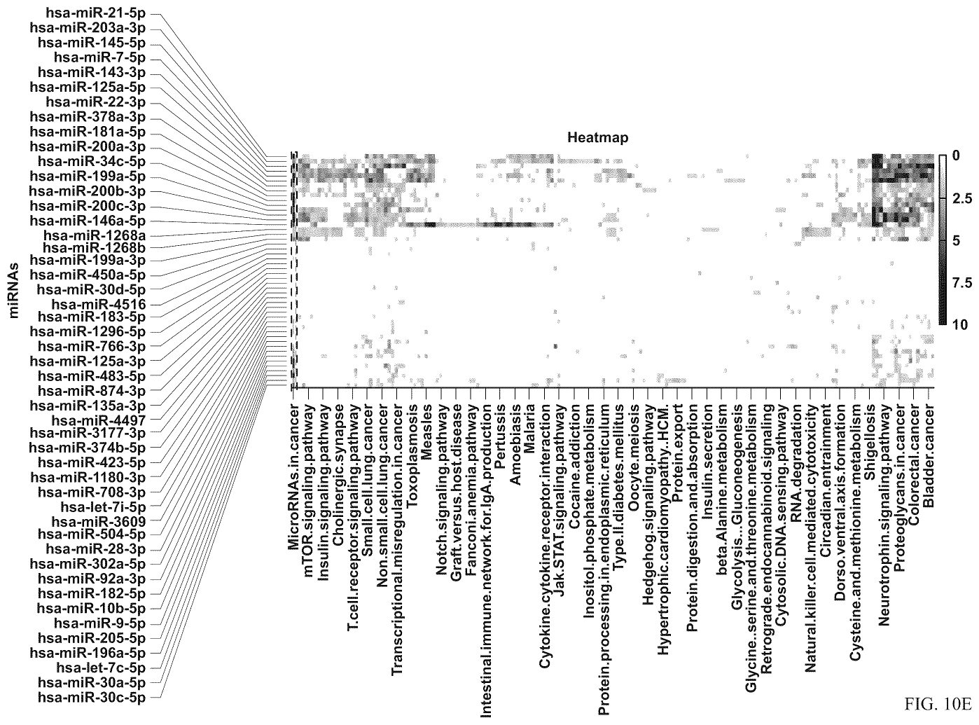

Genomic analysis was performed on exosome miRNA cargo from day 12 and day 21 exosomes using small RNA sequencing. Exosomes derived from day 21 neutrophils had higher counts of miRNA (˜15,000-20,000) than those of day 12 cells (˜4,500-5,000) (FIG. 10A). PCA plotting showed that the two groups form distinct clusters along principle component 1, with some variation within samples occurring in principle component 2 (FIG. 10B). The most expressed miRNAs within d12 EVs include miR-92a, miR-7, miR-101, miR-26a, miR-21, and miR-10b. Withing d21 EVs, the highest expressed miRNAs include miR-92a, miR-21, miR-20a, miR-26a, miR-10b, and miR-101. Heatmap analysis reveals further differences between the samples, with little overlap being observed between principle components in the two samples (FIG. 10C). Volcano plotting shows that 48 miRNAs were significantly downregulated in d12 vs. d21, 26 miRNAs were significantly upregulated in d12 vs. d21, and 234 miRNAs not being significantly different between the two samples (FIG. 10D). Heatmap analysis of miRNAs highly upregulated in day 21 exosomes shows that many of the upregulated miRNAs are most strongly correlated with cancer pathways and correlate with proliferative pathways (ex. mTOR, Notch, and Hedgehog) (FIG. 10E).

Differentially expressed genes are organized into Venn diagrams and compared to miRNAs known to be active in cancer and glioblastoma. 51 genes are expressed in both d12 and d21 EVs are active in cancer pathways, while d12 EVs uniquely express 8 genes active in cancer pathways and d21 EVs express 17 genes active in cancer pathways not expressed to d12 EVs (FIG. 11A). 12 miRNAs active in glioblastoma pathways are only expressed in d12 EVs, while 15 miRNAs active in glioblastoma pathways are expressed only in d21 EVs. 43 miRNAs active in glioblastoma pathways are expressed in d12 and d21 EVs (FIG. 11B). Of the 43 miRNAs active in glioblastoma expressed in both d12 and d21 EVs, genes upregulated in d21 EVs include miR-21, miR-30a, miR-182, miR-302b, miR-103a, miR-20a, miR-101, miR-30c, miR-191, and miR-25. Genes upregulated in d12 EVs compared to d21 EVs include miR-181a, miR-181b, miR-152, miR-378a, miR-126, miR-7, miR-146a, miR-143, miR-10b, and miR-148a (FIG. 11C).

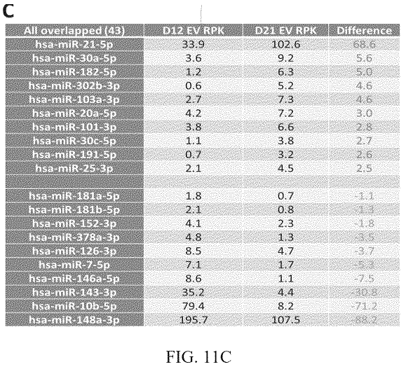

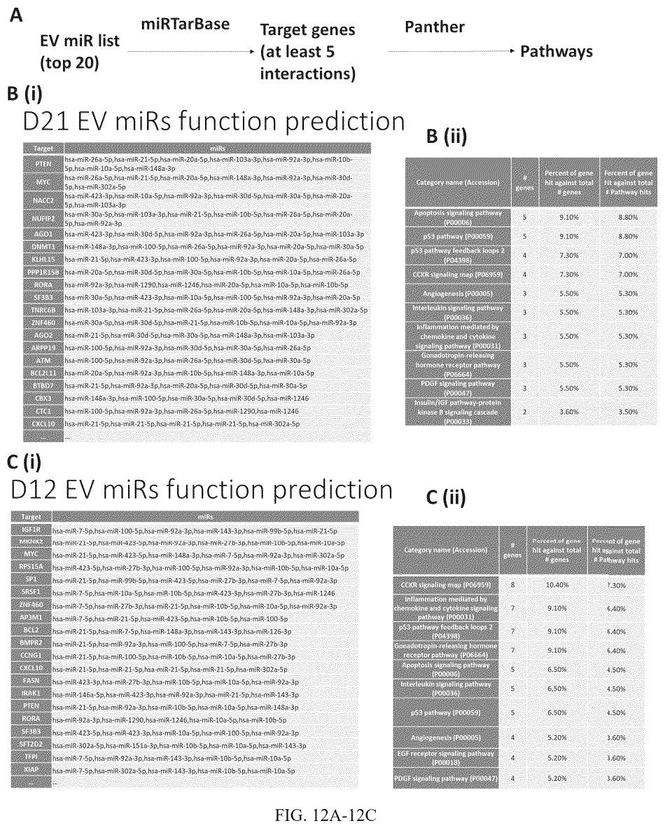

The function of both EV groups was carried out using the top 20 miRNAs held within each sample. The target genes of these miRNAs were identified using miRTarBase, which was then input to Panther to predict active pathway function. The top gene targets for d21 EVs include PTEN, MYC, NACC2, and NUFIP2 (FIG. 12A). The regulation of these genes correlates to function in apoptosis signaling, the p53 pathway, CCKR signaling, angiogenesis, interleukin signaling, and inflammation (FIG. 12A). The top gene targets for d12 EVs include IGF1R, MKNK2, MYC, RPS15A, SP1, SRSF1, and ZNF460 (FIG. 12B (i)). The corresponding function of d12 EVs includes CCKR signaling, inflammation, p53 pathway, apoptosis signaling, interleukin signaling, and angiogenesis (FIG. 12B (ii)). This analysis suggests that EVs from both d12 and d21 EVs will inhibit cancer progression via gene silencing mediated by their miRNA cargo.

### In Vitro CAR EV Testing

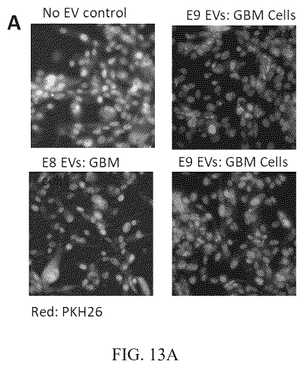

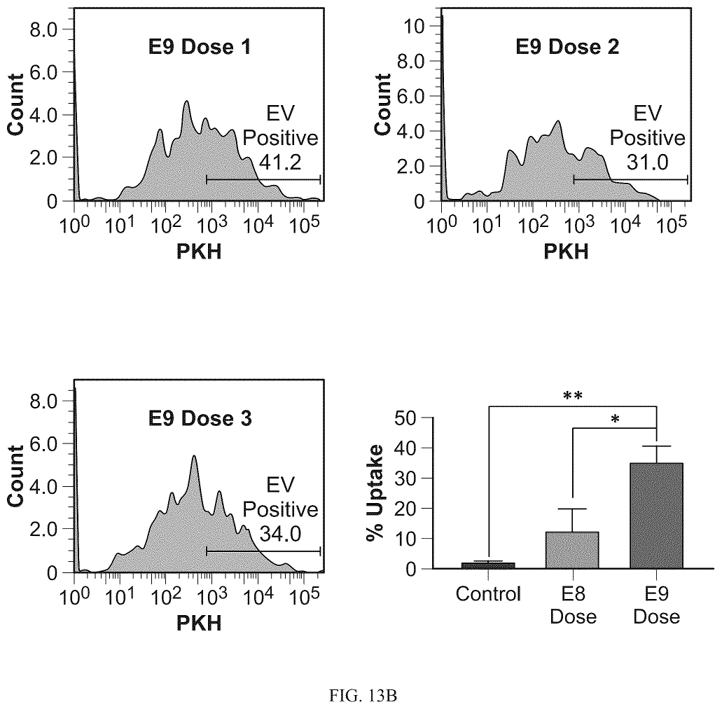

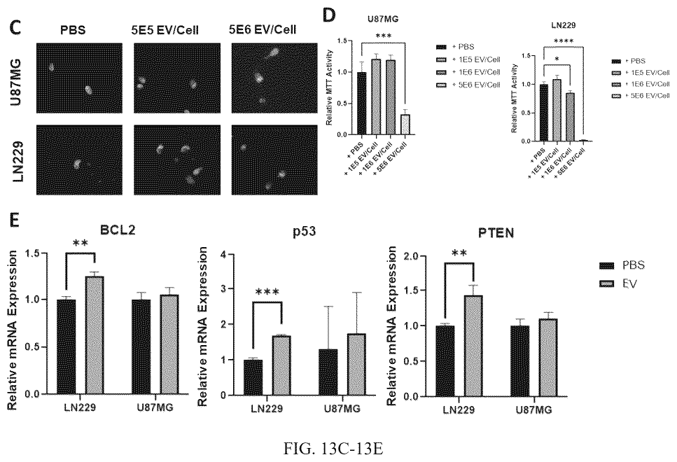

To confirm successful EV uptake occurs, CAR EVs were labelled with PKH27 dye and incubated with cells for 24 hours. Fluorescent imaging of cells treated with labelled EVs shows successful uptake at various EV doses (FIG. 13A). Flow cytometry analysis of cells treated with labelled EVs shows successful uptake of labelled EVs, with the percentage uptake of labelled EVs increasing with dose, showing uptake is dose-dependent (FIG. 13B). In addition to successful uptake, viability testing reveals that CAR-EVs have dose-dependent cytotoxic effects in both cell lines, with 5*106 EVs/cell can induce significant decreases in tumor viability (FIG. 13C).

To assess which pathways were active in mediating tumor cytotoxicity, quantitative PCR was performed. PCR for apoptotic genes revealed mixed results, with increased genomic expression of anti-apoptotic protein BCL2 and an increase in expression of pro-apoptotic transcription factor p53 mRNA in LN229 tumor cells. The mixed upregulation of pro and anti-apoptotic proteins still leading to tumor cell death is likely due to BCL2 expression being silenced by miRNAs present in CAR-EVs such as miR-21 and miR-148a. Other apoptotic proteins screened had no significant alteration in RNA expression (FIG. 13D). In addition, mRNA expression of tumor suppressor gene PTEN was tested given its heavy implication in CLTX-CAR genomic analysis. PCR revealed that PTEN mRNA was significantly upregulated in LN229 EV treated cells, suggesting that CLTX CAR EVs suppress tumor growth via inhibition of PI3K/AKT/mTOR pathway activation (FIG. 13D).

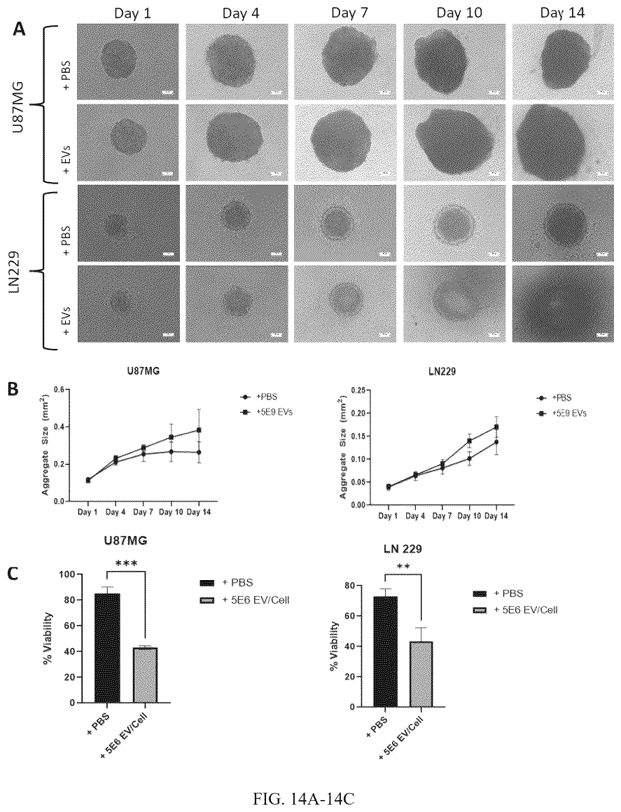

To further confirm the efficacy of CLTX-CAR Neu derived EVs, a 3D spheroid tumor model was used to evaluate EV cytotoxicity under conditions more like those seen in vivo. Over the course of 2 weeks of treatment, spheroid growth was monitored using microscopy imaging (FIG. 14A). An average increase in spheroid size was observed over time, with EV treated spheroids growing larger than PBS treated cells, though not significantly (FIG. 14B). Trypan blue staining revealed a significant decrease in tumor viability over the duration of 2 weeks (FIG. 14C). PKH26 labelling of EVs revealed that CAR EVs is capable of penetrating into tumor spheroids, confirming EV ability into the tumor microenvironment (FIG. 14D).

### Discussion

Glioblastoma multiforme (GBM) is the most commonly occurring tumor in the central nervous system, accounting for 80% of brain tumors. Current treatments for GBM include surgical resection, radiotherapy, chemotherapy, or a combination of these prove ineffective at treating GBM, with the median survival being 15 months after diagnosis. Work in gene editing and immunology has led to the development of chimeric antigen receptors (CARs), artificial proteins designed to illicit a strong immune response in the presence of a specific antigen.

CAR-based immunotherapy has been used successfully in clinical and research settings, enhancing immune cells tumor targeting and immune responsiveness. However, the limited lifetime of peripheral immune cells outside the body leaves the production of CAR engineered EVs limited when sourced from peripheral blood-derived immune cells. The use of CAR engineered immune cells derived from induced pluripotent stem cells addresses these issues, giving engineered cells at high yield and with higher ex vivo lifespan than immune cells derived from peripheral blood. iPSC derived CAR immune cells also illicit stronger immune responses to tumor cells than their peripheral blood counterparts. However, complications arising from off-target effects, overactivation, and tumor silencing still affect cells derived from both sources.

The cargo of CAR EVs secreted by iPSC-derived CAR neutrophils were analyzed herein and their cytotoxic abilities against in vitro models of glioblastoma. Immune-cell derived extracellular vesicles bypass excess cytokine release and tumor immune silencing via mechanisms like PD-1 expression while retaining the targeting and tumor cytotoxicity of their parent cells, overcoming some of the weaknesses CAR engineered cells present. Protein and miRNA cargo analysis of CAR EVs shows that as iPSCs differentiate into neutrophils, they pack proteins and miRNAs involved in apoptotic, proliferative, and inflammatory pathways into secreted EVs, suggesting a shift towards an anti-cancer phenotype. Increases in miRNA that dictate tumor suppression and apoptosis led to an increase in gene expression of mRNA related to these pathways, such as PTEN and p53. This suggests that inhibition of PI3K/AKT/MTOR signaling via miRNAs such as miR-148b and miR-30a and activation of p53 signaling via miRNAs such as miR-26a and miR-375 play roles in the cytotoxic effects of CLTX CAR neutrophil EVs. These pathways are heavily implicated in tumor progression as well, with regulation of the PI3K/AKT/MTOR axis being a common therapeutic target in cancer therapy. Likewise, restoring the function of the p53 tumor suppression pathway is a well-studied cancer therapy strategy. It is theorized that the miRNA cargo of the CLTX CAR EVs acts on both pathways to mediate their tumor suppressive properties.

Extracellular vesicles derived from immune cells have displayed the same cytotoxic effects against cancer cells as their parent cells. CAR displaying immune cell-derived EVs also share this trend. For instance, EVs derived from CAR-engineered T cells have been used to treat non-small cell lung cancer, breast cancer, and acute leukemia. In addition, compared to their unmodified counterparts, CAR-EVs are more efficiently trafficked into the tumor microenvironment, taken up at greater number, and induce stronger cytotoxic effects. While the exact reasoning behind this increase in efficacy being known, it is speculated to result from differences in protein and miRNA cargo due to gene editing or the greater specificity of EV uptake because of the targeting ability granted by the chimeric antigen receptor. Future work will focus on better understanding the mechanisms behind CAR EV mediated cytotoxicity and increasing the homogeneity of CLTX-CAR neutrophil EVs, as neutrophil EVs can vary based upon neutrophil function and age at time of secretion.

### CONCLUSION

In conclusion, this study provides an in-depth analysis of the protein and RNA cargo held within novel, CAR displaying extracellular vesicles derived from neutrophils differentiated from iPSCs. It also displays the cytotoxicity of these EVs against multiple glioblastoma lines, acting as therapeutic carriers to specifically target tumor cells to deliver cell-derived proteins and miRNAs to induce apoptosis. The concept of using CAR engineering to grant EVs a targeting ability is easily translatable to other cancer lines, providing an effective alternative to CAR engineered cells in cancer therapy.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

What is claimed is:

1. An engineered neutrophil extracellular vesicle (NPEV) comprising:
(a) a cancer- or tumor cell-targeting Chimeric antigen receptor (CAR);
(b) a therapeutic composition; and
(c) at least one miRNA.

2. The engineered NPEV of claim 1, wherein the engineered NPEV is derived from a parent CAR-induced pluripotent stem cell (iPSC).

3. The engineered NPEV of claim 1, wherein the engineered NPEV does not express Programmed Cell Death Protein 1 (PD-1).

4. The engineered NPEV of claim 2, having increased anti-cancer or anti-tumor effects compared to the parent CAR-iPSC.

5. The engineered NPEV of claim 2, having lower toxicity compared to the parent CAR-iPSC.

6. The engineered NPEV of claim 1, wherein the at least one miRNA comprises at least one miRNA targeting Lamin B2 (LMNB2).

7. The engineered NPEV of claim 1, wherein the at least one miRNA is selected from miR-3148, miR-4698, miR-3133, miR-5700, or a combination thereof.

8. The engineered NPEV of claim 1, wherein the at least one miRNA comprises miR-3133.

9. The engineered NPEV of claim 1, wherein the at least one miRNA comprises at least one miRNA targeting Programmed Cell Death Ligand 1 (PD-L1).

10. The engineered NPEV of claim 1, wherein the at least one miRNA is selected from miR-3117, miR-5193, miR-4282, miR-548, or a combination thereof.

11. The engineered NPEV of claim 1, wherein the at least one miRNA comprises miR-5193.

12. The engineered NPEV of claim 1, wherein the at least one miRNA comprises two or more miRNAs.

13. The engineered NPEV of claim 1, wherein the at least one miRNA comprises dual miRNAs.

14. The engineered NPEV of claim 1, further comprising at least one additional miRNA.

15. The engineered NPEV of claim 1, further comprising at least one additional miRNA selected from miR-34a, miR-424, miR-7, miR-10b, miR-20a, miR-21, miR-22, miR-25, miR-26a, miR-30a, miR-30c, miR-34c, miR-92a, miR-101, miR-103a, miR-125a, miR-143, miR-145, miR-146a, miR-148b, miR-181a, miR-182, miR-183, miR-191, miR-199a, miR-203a, miR-302b, miR-375, miR-378a, or a combination thereof.

16. The engineered NPEV of claim 1, wherein the therapeutic composition is Chlorotoxin (CLTX).

17. The engineered NPEV of claim 1, wherein the NPEV has a size of between about 30 nanometers and about 200 nanometers.

18. A pharmaceutical composition comprising the engineered NPEV of claim 1, and a pharmaceutically acceptable carrier.

19. The pharmaceutical composition of claim 18, wherein the engineered NPEV is formulated in a dose of between about 1×10⁷ EVs/mL and about 1×10¹¹ EVs/mL.

20. A method of reducing or inhibiting growth of a cancer or a tumor in a subject, comprising administering to the subject an engineered NPEV of claim 1 or a pharmaceutical composition of claim 18.

21. The method of claim 20, wherein the cancer or tumor is selected from non-small cell lung cancer (NSCLC), breast cancer, acute leukemia, or glioblastoma (GBM).

22. The method of claim 20, wherein the engineered NPEV is administered by intravenous injection, intratumoral injection, or a combination thereof.
